(19) United States
(12) Patent Application Publication
(10) Pub. No.: US 2010/0233132 A1
(43) Pub. Date: Sep. 16, 2010

(54) FORMATION OF VASCULAR NETWORKS USING EMBRYONIC STEM CELLS

(75) Inventors: Lino da Silva Ferreira, Cambridge, MA (US); Sharon Gerecht-Nir, Brookline, MA (US); Robert S. Langer, Newton, MA (US); Gordana V. Vunjak-Novakovic, Belmont, MA (US)

Correspondence Address: CHOATE, HALL & STEWART LLP, TWO INTERNATIONAL PLACE, BOSTON, MA, 02110, US

(73) Assignee: MASSACHUSETTS INSTITUTE OF TECHNOLOGY, Cambridge, MA (US)

(21) Appl. No.: 12/300,419

(22) PCT Filed: May 25, 2007
(86) PCT No.: PCT/US2007/069809

§ 371 Date: Dec. 15, 2009

Related U.S. Application Data
(60) Provisional application No. 60/809,148, filed on May 25, 2006.

(51) Int. Cl. A61K 35/54; C12N 5/0735; C12N 5/07

(52) U.S. Cl. 424/93.7; 435/366

(57) ABSTRACT

In various aspects, provided are methods for providing CD34⁺ cells from embryoid bodies and stimulating these cells to give rise to endothelial-like and/or smooth muscle-like cells. In various embodiments are provided methods that produce endothelial-like and/or smooth muscle-like cells that have functionality and/or preserved genetic integrity. In various aspects, provided are tissue engineering constructs comprising endothelial-like and/or smooth muscle-like cells produced according to a method the present inventions.

### CROSS-REFERENCE TO RELATED APPLICATIONS

The present application claims the benefit of and priority to copending U.S. provisional application No. 60/809,148 filed May 25, 2006, the entire contents of which are herein incorporated by reference.

### GOVERNMENT RIGHTS

This invention was made with support from the National Institutes of Health under Grant Nos. HL060435, DE13023, and HL076485. The U.S. government may have certain rights in this invention.

### BACKGROUND OF THE INVENTION

The vascularization of tissue constructs remains a major challenge in regenerative medicine, as the diffusional supply of oxygen can support only 100-200 μm thick layers of viable tissue. The formation of a mature and functional vascular tube network includes communication between endothelial cells (ECs) and smooth muscle cells (SMCs). Isolating a population of human progenitor cells with potential for cell number expansion and differentiation into both ECs and SMCs with high efficiency could benefit the area of tissue engineering.

### SUMMARY OF THE INVENTION

The present inventions, in various aspects, relate to the preparation of cell populations from embryonic stem cells. Prior to further describing the present inventions, it may be helpful to an understanding thereof to set forth definitions of certain terms to be used herein.

“Biomolecules”: The term “biomolecules”, as used herein, refers to molecules (e.g., proteins, amino acids, peptides, polynucleotides, nucleotides, carbohydrates, sugars, lipids, nucleoproteins, glycoproteins, lipoproteins, steroids, etc.) whether naturally-occurring or artificially created (e.g., by synthetic or recombinant methods) that are commonly found in cells and tissues. Specific classes of biomolecules include, but are not limited to, enzymes, receptors, neurotransmitters, hormones, cytokines, cell response modifiers such as growth factors and chemotactic factors, antibodies, vaccines, haptens, toxins, interferons, ribozymes, anti-sense agents, plasmids, DNA, and RNA.

“Biocompatible”: The term “biocompatible”, as used herein is intended to describe materials that do not elicit an undesirable detrimental response in vivo.

“Biodegradable”: As used herein, “biodegradable” polymers are polymers that degrade fully (i.e., down to monomeric species) under physiological or endosomal conditions. In preferred embodiments, the polymers and polymer biodegradation byproducts are biocompatible. Biodegradable polymers are not necessarily hydrolytically degradable and may require enzymatic action to fully degrade.

Embryonic stem cells are described as “undifferentiated” when a substantial portion of stem cells and their derivatives in the population display morphological characteristics of undifferentiated cells, clearly distinguishing them from differentiated cells of embryonic or adult origin. Undifferentiated embryonic stem cells are easily recognized by microscopic view as cells with high nuclear/cytoplasm ratios and prominent nucleoli. Similarly, undifferentiated cells can be distinguished from differentiated cells by the expression of one or more of the following stem cell markers: SSEA-4, TRA-1-60, TRA-1-81, Nanog and alkaline phosphatase. Human embryonic stem cells also express surface antigens initially described in other stem cell populations such as AC133, c-kit (CD177), flt3 (CD135) and CD9 (Hoffman & Carpenter, Nature Biotechnology 2005, 23(6), 699-708).

“Vascular progenitor cells” refers to a population of cells that can generate progeny that are endothelial or smooth muscle precursors (such as angioblasts) or mature endothelial or smooth muscle cells, or hematopoietic precursor (such as erythroid colony forming units and megakaryocytes) or mature blood cells (such as erythrocytes and leukocytes). Vascular progenitor cells may express some of the phenotypic markers that are characteristic of the endothelial, smooth muscle and hematopoetic lineages. Vascular progenitor cells include EL, SML, and HL.

CD34⁺ cells refers to cells expressing CD34 antigen. This antigen is a single-chain transmembrane glycoprotein expressed in several cells including human hematopoietic stem and progenitor cells, vascular endothelial cells, embryonic fibroblasts and some cells in fetal and adult nervous tissue.

“Endothelial like cells” refers to cells that can themselves or whose progeny can differentiate into mature endothelial cells. These cells may, but need not, have the capacity to generate hematopoietic or smooth muscle cells.

“Smooth muscle-like cells” refers to cells that can themselves or whose progeny can differentiate into mature smooth muscle cells. These cells may, but need not, have the capacity to generate hematopoietic or endothelial cells.

“Hematopoetic-like cells” refers to cells that can themselves or whose progeny can form myeloid, erythroid, and/or megakaryocyte colonies as described in Eaves, et al., Atlas of Human Hematopoietic Colonies, 1995, StemCell Technologies, Vancouver; Coutinho, et al, in Hematopoiesis: A Practical Approach, Testa, et al, eds., 1993, Oxford Univ. Press, NY, pp 75-106, and Kaufman, et al., PNAS, 2001, 98:10716-10721.

“Growth Factors”: As used herein, “growth factors” are chemicals that regulate cellular metabolic and/or signaling processes, including but not limited to differentiation, proliferation, synthesis of various cellular products, and other metabolic activities. Growth factors may include several families of, chemicals, including but not limited to cytokines, eicosanoids, and differentiation factors.

“Polynucleotide”, “nucleic acid”, or “oligonucleotide”: The terms “polynucleotide”, “nucleic acid”, or “oligonucleotide” refer to a polymer of nucleotides. The terms “polynucleotide”, “nucleic acid”, and “oligonucleotide”, may be used interchangeably. Typically, a polynucleotide comprises at least three nucleotides. DNAs and RNAs are polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

“Polypeptide”, “peptide”, or “protein”: According to the present invention, a “polypeptide”, “peptide”, or “protein” comprises a string of at least three amino acids linked together by peptide bonds. The terms “polypeptide”, “peptide”, and “protein”, may be used interchangeably. Peptide may refer to an individual peptide or a collection of peptides. Inventive peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

“Polysaccharide”, “carbohydrate” or “oligosaccharide”: The terms “polysaccharide”, “carbohydrate”, or “oligosaccharide” refer to a polymer of sugars. The terms “polysaccharide”, “carbohydrate”, and “oligosaccharide”, may be used interchangeably. Typically, a polysaccharide comprises at least three sugars. The polymer may include natural sugars (e.g., glucose, fructose, galactose, mannose, arabinose, ribose, and xylose) and/or modified sugars (e.g., 2′-fluororibose, 2′-deoxyribose, and hexose).

“Small molecule”: As used herein, the term “small molecule” is used to refer to molecules, whether naturally-occurring or artificially created (e.g., via chemical synthesis), that have a relatively low molecular weight. Typically, small molecules are monomeric and have a molecular weight of less than about 1500 g/mol. Preferred small molecules are biologically active in that they produce a local or systemic effect in animals, preferably mammals, more preferably humans. In certain preferred embodiments, the small molecule is a drug. Preferably, though not necessarily, the drug is one that has already been deemed safe and effective for use by the appropriate governmental agency or body. For example, drugs for human use listed by the FDA under 21C.F.R. §§330.5, 331 through 361, and 440 through 460; drugs for veterinary use listed by the FDA under 21 C.F.R. .sctn. .sctn. 500 through 589, incorporated herein by reference, are all considered acceptable for use in accordance with the present invention.

“Bioactive agents”: As used herein, “bioactive agents” is used to refer to compounds or entities that alter, inhibit, activate, or otherwise affect biological or chemical events. For example, bioactive agents may include, but are not limited to, anti-AIDS substances, anti-cancer substances, antibiotics, immunosuppressants, anti-viral substances, enzyme inhibitors, neurotoxins, opioids, hypnotics, anti-histamines, lubricants, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, anti-depressants, anti-psychotic substances, anti-emetics, and imaging agents. In certain embodiments, the bioactive agent is a drug.

A more complete listing of bioactive agents and specific drugs suitable for use in the present invention may be found in “Pharmaceutical Substances: Syntheses, patents, applications” by Axel Kleemann and Jurgen Engel, Thieme Medical Publishing, 1999; the “Merck Index: An Encyclopedia of Chemicals, Drugs, and Biologicals”, Edited by Susan Budavari et al., CRC Press, 1996, and the United States Pharmacopeia-25/National Formulary-20, published by the United States Pharmcopeial Convention, Inc., Rockville Md., 2001, all of which are incorporated herein by reference.

“Tissue”: as used herein, the term “tissue” refers to a collection of cells of one or more types combined to perform a specific function, and any extracellular matrix surrounding the cells.

“Passaging”: as used herein the term passaging refers to the transfer of cells from one culture vessel to another. This usually involving the subdivision of a proliferating cell culture that has reached confluence. This process is also sometimes referred to as subculturing or splitting of a cell culture.

### Abbreviations

For ease and conciseness of description, the following abbreviations are used herein.

ECs—endothelial cells

SMCs—smooth muscle cells

ESCs—embryonic stem cells

EBs—embryoid bodies

ac-LDL—acetylated low-density lipoprotein

FACS—fluorescence-activated cell sorting

PECAM1 platelet endothelial cell-adhesion molecule-1

hESC—human embryonic stem cell

FBS—fetal bovine serum

HUVEC—human umbilical vein endothelial cell

hVSMC—human vascular smooth muscle cell

PDGFBB—Platelet Derived Growth Factor BB

EL—endothelial-like cell

SML—smooth muscle-like cell

In various aspects, the present inventions provided methods for providing a population of endothelial-like and/or smooth muscle-like from a population of stem cells. In various aspects, provided methods that produce endothelial-like and/or smooth muscle-like cells that have functionality and/or preserved genetic integrity. In various aspects, provided are tissue engineering constructs comprising endothelial-like and/or smooth muscle-like produced according to a method the present inventions disposed in and/or on a support substrate.

In various embodiments, the provided are methods for obtaining a population of differentiated cells from a population of stem cells, comprising the steps of: contacting a population of stem cells with a differentiation medium to form a population of embryoid bodies; extracting from the population of embryoid bodies at least a portion of the cells expressing a vascular progenitor cell marker to provide a population of vascular progenitor cells; contacting the a population of vascular progenitor cells with one or more growth factors such that at least a portion of the population of vascular progenitor cells differentiate into one or more of endothelial-like cells and/or smooth muscle-like cells. In various embodiments, the vascular progenitor cell marker is the CD34 marker and the vascular progenitor cells are CD34⁺ cells.

In various embodiments, the methods of the present inventions provide methods for extracting from a population of embryoid bodies greater than about 5%, greater than about 10%, and/or greater than about 15% of the cells expressing a vascular progenitor cell marker from the population of embryoid bodies. In various embodiments, the vascular progenitor cell marker is CD34.

In various embodiments, the one or more growth factors comprise VEGF₁₆₅ and the differentiated cells comprise endothelial-like cells. In various embodiments, the one or more growth factors comprise PDGF_{BB} and the differentiated cells comprise smooth muscle-like cells. In various embodiments, the population of vascular progenitor cells is contacted with concentration of growth factor that is greater than about 30 ng/ml, greater than about 50 ng/ml, and/or in the range between about 30 ng/ml to about 100 ng/ml.

In various embodiments, the one or more growth factors comprise VEGF₁₆₅ or PDGF are in contact with vascular progenitor cells preferably for 8-15 days or most preferably for 15-30 days to differentiate into endothelial or smooth muscle cells, respectively.

In various embodiments, the methods of the present invention provide a population of endothelial-like cells and/or smooth muscle-like cells wherein the karyotype of the endothelial-like cells and/or is substantially preserved relative to the karyotype of the stem cells from which they were derived. In various embodiments, it is preferred that the step of contacting a population of stem cells with a differentiation medium comprises passaging the cells less than about 50 times, and/or less than about 30 times. In various embodiments, the karyotype of the cells is substantially preserved when greater than about 9 in 10 cells have a preserved karyotype. In various embodiments, the karyotype of the cells is substantially preserved when greater than about 19 in 20 cells have a preserved karyotype.

In various embodiments, the present inventions provide methods that provide functional SML cells. For example, in various embodiments, provided are SML that can contract and/or relax in response to a stimuli. In various embodiments, provided are cell populations where at least 10%, at least 25%, at least 50%, at least 75%, and/or at least 90% of the smooth muscle-like cells are functional.

In various embodiments, the present inventions provide methods that provide functional EL cells. For example, in various embodiments, provided are EL that can form extensive vascular networks when they are seeded on top of matrigel.

In various embodiments, present inventions provide methods for promoting the development of vascular tissue using embryonic stem cells, comprising: providing a first population of embryonic stem cells; contacting the first population with a cell release agent, e.g. type IV collagenas, to extract a population of released cells; culturing the released cells (e.g., embryoid bodies) in a differentiation medium; isolating those cells expressing a vascular progenitor cell marker to produce a population of vascular progenitor cells; and culturing the vascular progenitor cells under predetermined conditions to cause their differentiation into endothelial-like cells and/or smooth muscle-like cells.

In various embodiments, the methods of the present inventions further comprise seeding the differentiated cells onto a three dimensional cell support substrate, such as, e.g., Matrigel™.

In various aspects, the present inventions provide an implantable construct comprising a cell support substrate and a population of endothelial-like and/or smooth muscle-like cells prepared according to a method of the present inventions. In various aspects, the present inventions provide an implantable construct comprising a cell support substrate, a population of vascular progenitor cells and one or more growth factors at a concentration of greater than about 30 ng/ml, greater than about 50 ng/ml, and/or in the range between about 30 ng/ml to about 100 ng/ml.

In various embodiments of the methods, populations and/or constructs of the present inventions the vascular progenitor cells are CD34⁺ cells and the vascular progenitor cell marker is CD34. In various embodiments, of the methods, populations and/or constructs of the present inventions having one or more growth factors, one or more of the growth factors is provided at a concentration of greater than about 30 ng/ml, greater than about 50 ng/ml, and/or in the range between about 30 ng/ml to about 100 ng/ml.

In various embodiments of the methods, populations and/or constructs of the present inventions the stem cells are embryonic stem cells, and in various embodiments the embryonic stem cells are human embryonic stem cells.

In various aspects and embodiments of the present inventions, vascular progenitor cells, e.g., CD34⁺, are extracted from a population of embryoid bodies. For example, a variety of approaches can be used to isolate CD34⁺ cells. For example, in various embodiments and examples CD34⁺ cells were isolated from human embryoid bodies by the use of magnetic beads containing the antibody anti-human CD34 clone AC136 (commercially available at Miltenyi Biotec, Germany) which recognizes a class III epitope of the CD34 antigen.

### BRIEF DESCRIPTION OF THE DRAWINGS

The foregoing and other aspects, embodiments, and features of the present inventions can be more fully understood from the following description in conjunction with the accompanying drawings.

The patent or application file contains at least one drawing executed in color. Copies of this patent or patent application publication with color drawing(s) will be provided by the Office upon request and payment of the necessary fee.

FIGS. 1A-B depict data of Example 1 regarding expression of vascular and undifferentiated stem cell markers in hES cells. FIG. 1A: Flow cytometric analysis of undifferentiating and vascular markers on undifferentiated hESCs. Percent of positive cells were calculated based in the isotype controls (grey plot) and are shown in the histogram plots. Values indicate average±SD, from 3 independent experiments. FIG. 1B Fluorescent immunostaining for CD34 (×25), SM-MHC (×25) and α-SMA (×40).

FIGS. 2A-B depict data of Example 1 regarding expression of vascular and undifferentiated stem cell markers during hESCs differentiation through EBs. FIG. 2A present summary of flow cytometric analysis for: (FIG. 2A.1) the expression of undifferentiating markers and vascular markers in EBs grown in differentiation medium containing KO-SR (black columns) or FBS (white columns), for 6 or 10 days; (FIG. 2A.2) the time-course expression of KDR/Flk-1 (squares), CD34 (circles) and PECAM1 (triangles) in EBs grown in differentiation medium containing KO-SR; (FIG. 2A.3) the expression of CD34 and co-expression with different markers, in EBs grown in differentiation medium containing KO-SR (black columns) or FBS (white columns), for 10 days. In all graphs, values indicate average±SD, from 3 independent experiments. The notations * and ** denote statistical significance (P<0.05 and P<0.01, respectively). FIG. 2B confocal microscopy of stained 10-day old human EBs grown in differentiation medium containing FBS. CD34⁺[(B.1, ×25), (B.2, ×40)] and PECAM1⁺ (B.3, ×25) cells forming vascular networks along the EBs.

FIGS. 3A-I depict data of Example 1 regarding isolation and differentiation of CD34⁺ cells. FIG. 1A schematically depicts scheme for the isolation and differentiation of CD34⁺ cells. Bar in microscope image corresponds to 500 p.m. FIGS. 3B-I depict FACS analysis of HUVEC (FIG. 3B), hVSMC (FIG. 3C), and CD34⁺ cells isolated from EBs grown in differentiation medium with FBS for 10 days and further differentiated in EGM-2 medium (FIGS. 3E,H), EGM-2 medium supplemented with 50 ngmL⁻¹ VEGF₁₆₅ (FIGS. 3D,G), or EGM⁻² medium supplemented with 50 ngmL⁻¹ PDGFBB (FIGS. 3F,I), for 1 passage (FIGS. 3D,E,F, 10-15 days after cell seeding) or 3 passages (FIGS. 3G,H,I, ca. 28 days after cell seeding). In all graphs, percent of positive cells were calculated based in the isotype controls (grey plot) and are shown in each histogram plot.

FIGS. 4A-D depict data of Example 1 regarding characterization of hES-derived endothelial cells and smooth muscle cells grown in culture. FIG. 4A EL cells have cobblestone morphology (FIG. 4A.1; light microscopy, bar corresponds to 50 μm), they show VE-cadherin at cell-cell junctions (FIG. 4A.2, ×40), and vWF in the cell cytoplasm (FIG. 4A.3, ×40), as shown by immunoflurescence staining. These cells have the ability to uptake ac-LDL (FIG. 4A.4, ×40) and to form cords when placed in Matrigel for 24 h (FIG. 4A.5; bar corresponds to 50 μm). FIG. 4B SML cells exhibit spindle-shaped morphology (FIG. 4B.1; light microscopy, bar corresponds to 50 μm) and highly express smooth muscle markers including α-SMA (FIG. 4B.2, ×125), SM-MHC (FIG. 4B.3, ×125) and calponin (FIG. 4B.4, ×125), as shown by immunofluorescence staining. These cells showed limited ability to form cords when placed in Matrigel for 24 h (FIG. 4B.5; bar corresponds to 50 μm). FIG. 4C presents data on the RT-PCR analysis for endothelial and smooth muscle cell markers in CD34⁺ cells differentiated in EGM-2 (column 1), EGM-2 supplemented with 50 ng mL⁻¹ VEGF₁₆₅ (column 2), and EGM-2 supplemented with 50 ng mL⁻¹ PDGF_{BB} (column)) Ang1, Ang2 and Cald are abbreviations for angiopoietin1, angiopoietin2 and caldesmon. FIG. 4D depicts transmitted electron microscopy images of cord sections formed by EL cells in Matrigel, showing lumen (Lu) formation (FIG. 4D.1). The cells present Weibel-Palade-like bodies (FIG. 4D.2, arrow) in the cytoplasm and form tight intercellular junctions (FIG. 4D.2, arrowheads). Bar corresponds to 0.47 μm.

FIGS. 5A-D depict data of Example 1 regarding transplantation of EL or SML cells in Nude mice. Matrigel alone (FIG. 5C), or matrigel containing EL cells (FIG. 5A), or a mixture of EL and SML cells (3:1) (FIG. 5C) was injected subcutaneously in the dorsal region of the nude mice (n=3, for each condition). After 28 days, the implants were removed, fixed and processed for immunohistochemistry. FIG. 5A: the implants with EL cells show microvessels that are immunoreactive for UEA-1 FIG. 5A.1 and FIG. 5A.2), anti-human PECAM1 (inset in FIG. 5A.1, ×40), and anti-human collagen type IV (FIG. 5A.3, ×64), and, in some cases, they also have mouse blood cells in their lumen (FIG. 5A.2). These microvessels are not reactive for anti-human α-SMA (FIG. 5A.4). FIG. 5B: the constructs with a mixture of EL and SML cells exhibit microvessels that are immunoreactive for anti-human PECAM1 (FIG. 5B.1), anti-human collagen type IV (FIG. 5B.2, ×64) and UEA-1 (FIG. 5B.3). The microvessels presented either an empty lumen (FIG. 5B.3, open arrowhead) or a lumen with mouse blood cells (FIG. 5B.3, closed arrowhead). α-SMA⁺ cells were observed inside of the matrigel and in some cases they formed small tubules (FIG. 5B.4). In the periphery of the matrigel (inset in FIG. 5B.4), α-SMA⁺ cells surrounded human ECs and formed microvessels carrying mouse blood. Bar represents 50 μm. FIG. 5C: hematoxylin/eosin staining of Matrigel construct without cells showing minimal mouse cell invasion in the regions where the matrigel did not degrade and mouse microvessels at the periphery of the implant (arrows). FIG. 5D: counts of human type IV collagen immunoreactive annular structures per five random high-power fields. The notation * and ** denote statistical significance, respectively, of P<0.005 and P<0.001.

FIGS. 6A-D depict data of Example 1 regarding FACS analysis of endothelial and smooth muscle cell markers in differentiated CD34⁺ cells isolated from H13 cell line. The cells were isolated from EBs grown in differentiation medium containing FBS for 10 days and then cultured on EGM-2 medium supplemented with 50 ng mL⁻¹ of VEGF₁₆₅ (FIGS. 6A,B), EGM-2 medium (FIG. 6C) or EGM-2 medium supplemented with 50 ng mL⁻¹ of PDGF_{BB} (FIG. 6D), for 1 (FIG. 6A) or 3 passages (FIGS. 6B,C,D). Percent of positive cells were calculated based in the isotype controls (grey plots) and are shown in each histogram plot.

FIG. 7 depicts data of Example 1 regarding expression of endothelial markers in differentiated CD34⁻ cells. FACS analysis of CD34⁻ cells isolated from EBs grown in differentiation medium with FBS for 10 days and further differentiated in EGM-2 medium supplemented with 50 ng mL⁻¹ VEGF₁₆₅ (A), for 1 passage (10-15 days after cell seeding). In all graphs, percent of positive cells were calculated based in the isotype controls (grey plot) and are shown in each histogram plot.

FIGS. 8A-B depict data of Example 1 regarding the cord-like structures formed by differentiated CD34⁺ cells (isolated from H13 cell line) on Matrigel. CD34⁺ cells differentiated on EGM-2 medium supplemented with 50 ng mL⁻¹ VEGF₁₆₅ form continuous and complex cords after their seeding on Matrigel for 24 h. Bar corresponds to 400 and 100 μm in FIG. 8A and 8B, respectively.

FIGS. 9A-B depict data of Example 1 regarding the transplantation of SML cells in nude mice. FIG. 9A: the constructs with SML cells stained positively for α-SMA; bar corresponds to 50 μm. FIG. 9B: the lumen of the microvessels was immunoreactive for anti-human collagen type IV (^{×}20).

FIGS. 10A-B depict data of Example 2 regarding expression of vascular and undifferentiated stem cell markers in hESCs. FIG. 10A: flow cytometric analysis of undifferentiated and vascular markers on undifferentiated hESCs. Percent of positive cells were calculated based in the isotype controls (grey plot) and are shown in the histogram plots. Values in histogram plots indicate average±SD from 3 independent experiments. FIG. 10B: gene analysis for vascular markers on undifferentiated hESCs.

FIGS. 11A-B depict data of Example 2 regarding expression of vascular and undifferentiated stem cell markers during hESCs differentiation through EBs. FIG. 11A presents a summary of flow cytometric analysis for: (FIG. 11A.1) the expression of undifferentiating markers and vascular markers in hESCs (grey columns) and EBs grown in differentiation medium containing KO-SR (black columns) or FBS (white columns) for 10 days; (FIG. 11A.2) the time-course expression of KDR/Flk-1 (□), CD34 (◯) and PECAM1 (Δ) in EBs grown in differentiation medium containing KO-SR. In all graphs, values indicate average±SD from 3 independent experiments, the notations *, ** and *** denote statistical significance of P<0.05, P<0.01 and P<0.001, respectively. FIG. 11B: confocal microscopy of stained 10-day old human EBs grown in differentiation medium containing FBS. CD34⁺ and PECAM1⁺ cells forming vascular networks along the EBs (FIG. 11B.1, ×25). Bar corresponds to 50 μm. Quantification of EBs that stained for PECAM1 and CD34 (FIG. 11B.2). At least 100 EBs were scored (average±SD, n=3). The notation * denotes statistical significance (P<0.05).

FIG. 12 depicts data of Example 2 regarding the expression of CD34 and PECAM1 in EBs grown in differentiation medium with FBS for 10 days, as assessed by FACS analysis. Values indicate average±SD, from 3 independent experiments.

FIGS. 13A-C depict data of Example 2 regarding the isolation and characterization of CD34⁺ cells. FIG. 13A schematically depicts a scheme for the isolation and differentiation of CD34⁺ cells. Bar corresponds to 500 um. FIG. 13B: phenotypic analysis of CD34⁺ cells after MACS separation. Values within dot plots indicate percentage of cells in respective quadrants. FIG. 13C: gene analysis of CD34⁺ cells after MACS separation.

FIGS. 14A-C depict data of Example 2 regarding endothelial and smooth muscle cell differentiation of CD34⁺ cells. FIG. 14A depicts FACS analysis of HUVEC and CD34⁺ cells isolated from EBs grown in differentiation medium with FBS for 10 days and further differentiated in EGM-2 medium, EGM-2 medium supplemented (as noted above columns of data plots) with 50 ngmL⁻¹ VEGF₁₆₅, or EGM-2 medium supplemented with 50 ngmL⁻¹ PDGF_{BB} for 1 passage (1P; 10-15 days after cell seeding) or 3 passages (3P; ca. 28 days after cell seeding). In all graphs, the percents of positive cells were calculated based in the isotype controls (grey plot) and are shown in each histogram plot. FIG. 14B depicts Western blot analysis for CD34⁺ cells differentiated in EGM-2 medium, EGM-2 medium supplemented with 50 ngmL⁻¹ VEGF₁₆₅, or EGM-2 medium supplemented with 50 ngmL⁻¹ PDGF_{BB} for 3 passages. HUVECs and SMCs are included for reference. GADPH was used as standard. FIG. 14C depicts relative band density for vascular markers using GADPH as a control protein.

FIGS. 15A-C depict data of Example 2 regarding the FACS analysis of endothelial and smooth muscle cell markers in differentiated CD34⁺ cells isolated from H13 cell line. The cells were isolated from EBs grown in differentiation medium containing FBS for 10 days and then cultured in EGM-2 medium supplemented with 50 ngmL⁻¹ of VEGF₁₆₅ (FIGS. 15A,B) or PDGF_{BB} (FIG. 15C), for 1 (FIG. 15A) or 3 passages (FIGS. 15B,C). Percent of positive cells were calculated based in the isotype controls (grey plots) and are shown in each histogram plot.

FIGS. 16A-B depict data of Example 2 regarding the expression of endothelial markers in differentiated CD34⁻ and CD34⁺ cells. FIG. 16A FACS analysis of CD34⁻ cells isolated from EBs grown in differentiation medium with FBS for 10 days and further differentiated in EGM-2 medium supplemented with 50 ngmL⁻¹ VEGF₁₆₅ (FIG. 16A), for 1 passage (10-15 days after cell seeding). In all graphs, the percents of positive cells were calculated based in the isotype controls (grey plot) and are shown in each histogram plot. FIG. 16A FACS analysis of CD34⁺ cells isolated from EBs grown in differentiation medium with FBS for 10 days and further differentiated in EGM-2 medium (FIG. 16B.1) or EGM-2 medium supplemented with 50 ngmL⁻¹ PDGF_{BB} (FIG. 16B.2), for 1 passage (10-15 days after cell seeding).

FIGS. 17A-B depict data of Example 2 regarding the karyotyping analyses of H13 (A) and H9 (B) cell lines. In H13 cells the karyotype obtained was 46, XY and is characteristic of a chromosomally normal male. In H9 cells the karyotype obtained was 46, XX and is characteristic of a chromosomally normal female. Cells were prepared and analysed as previously described (Cowan, C. A. New England Journal of Medicine 2004; 350:1353-1356). Approximately 20 metaphases spreads were counted and 5 metaphases analysed for each sample. Karyotyping analysis was performed by the Dana Faber/Harvard Cancer Research Center, Cytogenetics Laboratory, Cambridge, Mass.

FIGS. 17C-D depict data of Example 2 regarding karyotyping analyses of CD34⁺ cells differentiated in VEGF (FIG. 17C) or PDGF (FIG. 17D) supplemented media for three passages. In both differentiated cells the karyotype obtained was 46, XX, and no clonal aberrations were observed in 20 cells examined.

FIGS. 18A-E depict data of Example 2 regarding characterization of hES-derived endothelial cells and smooth muscle cells grown in culture. FIG. 18A: EL cells have cobblestone morphology (FIG. 18A.1; light microscopy, bar corresponds to 50 μm), they show VE-cadherin at cell-cell junctions (FIG. 18A.2, ×40), vWF in the cell cytoplasm (FIG. 18A.3, ×40) and have the ability to uptake ac-LDL (FIG. 18A.4, ×40), as shown by immunoflurescence staining. FIG. 18B: SML cells exhibit spindle-shaped morphology (FIG. 18B.1; light microscopy, bar corresponds to 50 μm) and highly express smooth muscle markers including α-SMA (FIG. 18B.2, ×40), SM-MHC (FIG. 18B.3, ×40) and calponin (FIG. 18B.4, ×40), as shown by immunofluorescence staining. FIG. 18C: EL cells form cords when placed in Matrigel for 24 h (FIG. 18C.1) while SML cells showed limited ability to form them during the same period of time (FIG. 18C.2). In both figures bar corresponds to 50 μm. The cord length (FIG. 18C.3) and branching points (FIG. 18C.4) on the cord-like structures formed by EL is statistically higher than the values found for SML cells during 24 or 48 h. The counts were performed using an objective of 10×. Results are average±SD, n=4. * denote statistical significance (P<0.001). FIG. 18D depicts transmission electron microscopy images of cord sections formed by EL cells in Matrigel, showing lumen (Lu) formation (FIG. 18D.1). The cells present Weibel-Palade-like bodies (FIG. 18D.2, arrow) in the cytoplasm, and form tight intercellular junctions (FIG. 18D.2, arrowhead). Bar corresponds to 0.47 μm. FIG. 18E RT-PCR analysis for endothelial and smooth muscle cell markers in CD34⁺ cells differentiated in EGM-2 (column 1), EGM-2 supplemented with 50 ngmL⁻¹ VEGF₁₆₅ (column 2) and EGM-2 supplemented with 50 ngmL⁻¹ PDGF_{BB} (column 3). Ang1, Ang2 and Cald are abbreviations for angiopoietin1, angiopoietin2 and caldesmon, respectively.

FIGS. 19A-B depict data of Example 2 regarding the characterization of HUVECs and human vascular smooth muscle cells (hVSMCs). FIG. 19A: HUVEC cells show vWF (×125), have the ability to uptake ac-LDL (×125) and present Weibel-Palade bodies (arrow) in the cytoplasm as shown by electron microscopy. Bar corresponds to 0.47 μm and in the inset 0.26 μm. FIG. 19B: hVSMCs express SM-MHC (×40), α-SMA (×40) and calponin (×40).

FIGS. 20A-B depict data of Example 2 regarding the ability of SML cells to contract to carbachol as hVSMCs. FIG. 20A presents data on the % contraction of SML cells and hVSMC. SML cells cultured for 3 passages were washed and contraction was induced by incubating these cells with 10⁻⁵M Carbachol in DMEM medium for 30 min. (FIG. 20A.1). Contraction was calculated by the difference of cell area at time zero and time 30 minutes. Bright-field images (×10 or ×20) were used for this purpose. In a separate experiment, the cells were induced to relax by incubation with 10⁻⁴M atropine in DMEM for 1 h and then induce to contract with 10⁻⁵ M Carbachol (FIG. 20A.2). Contraction was calculated as before. hVSMCs (3^{rd} passage) were used as controls. In B, morphological changes when SML were stimulated by carbachol (FIG. 20B.1 and FIG. 20B.2: before and after treatment, respectively).

FIGS. 21A-B depict data of Example 2 regarding the cord-like structures formed by differentiated CD34⁺ cells (isolated from H13 cell line) on Matrigel. CD34⁺ cells differentiated on EGM-2 medium supplemented with 50 ngmL⁻¹ VEGF₁₆₅ form continuous and complex cords after their seeding on matrigel for 24 h. Bar corresponds to 400 and 100 μm in FIG. 21A and FIG. 21B, respectively.

FIG. 22 depicts data of Example 2 regarding the formation of vessels in Matrigel implants that support blood flow. EL and SML cells alone or EL cells mixed with SML cells were suspended in Matrigel and injected subcutaneously in the dorsal region of a balb/c nude mice. After 28 days, the mice were injected intravenously, through the tail vein, with 0.2 mL of PBS containing 50 mg/mL of FITC-dextran (MW 145 kDa). Animals were sacrificed 10 min following injection and the Matrigel implant removed and imaged. Microvessels that support blood flow were observed in Matrigel implants containing EL (×10), SML (×10) or a mixture of EL and SML (×10) cells, but rarely in Matrigel without cells.

FIGS. 23A-D depict data of Example 2 regarding the transplantation of EL or SML cells in Nude mice. Matrigel alone (FIG. 23A), or matrigel containing EL cells (FIG. 23B), or a mixture of EL and SML cells (3:1) (FIG. 23C) was injected subcutaneously in the dorsal region of the nude mice (n=3, for each condition). After 28 days, the implants were removed, fixed and processed for histological evaluation. FIG. 23A: hematoxylin/eosin staining of Matrigel construct without cells showing mouse microvessels at the periphery of the implant (arrows) but not within Matrigel. FIG. 23B: the implants with EL cells show microvessels that are reactive for human UEA-1 (FIG. 23B.1 and 23B.2), anti-human PECAM1 (FIG. 23B.3, ×64) and anti-human collagen type IV (FIG. 23B.4, ×64), and in some cases they have mouse blood cells in their lumen (FIG. 23B.2). These microvessels are not reactive for anti-human α-SMA (FIG. 23B.5). FIG. 23C: the constructs with a mixture of EL and SML cells show microvessels that are reactive for human UEA-1 (FIG. 23C.1), anti-human PECAM1 (FIG. 23C.2, ×25) and anti-human collagen type IV (FIG. 23C.3, ×64). The microvessels presented either an empty lumen (FIG. 23C.1, open arrowhead) or a lumen with mouse blood cells (FIG. 23C.1, closed arrowhead). α-SMA⁺ cells were observed inside of the matrigel and in some cases they formed small tubules (FIG. 23C.4). In the periphery of the matrigel (FIG. 23C.5), α-SMA⁺ cells surrounded human ECs and formed microvessels carrying mouse blood. In all figures bar represents 50 μm. FIG. 23D: counts of human type IV collagen immunoreactive annular structures per five random high-power fields.

FIGS. 24A-B depict data of Example 2 regarding the transplantation of EL and SML cells in balb/c nude mice. Negative controls for samples of Matrigel containing EL cells (FIG. 24A), or a mixture of EL and SML cells (3:1) (FIG. 24B). Negative controls for UEA-1 (FIG. 24A.1 and FIG. 24B.1), collagen type IV (FIG. 24A.2 and FIG. 24B.2, ×64), PECAM1 (FIG. 24B.3, ×25), and α-SMA (FIG. 24B.4, ×64). Bar represents 50 μm. In case of UEA-1, the negative control was prepared according to the manufacturer specifications, i.e., by inhibiting the UEA-1 with 100 mM L-(−)-fucose (Sigma) in 10 mM HEPES, pH 7.5 containing 0.15 M NaCl, for 30 min, at room temperature.

FIGS. 25A-C depict data of Example 2 regarding the transplantation of EL and SML cells in balb/c nude mice. FIG. 25A: the constructs with EL and SML cells contained regions that stained positively for human β₂-microglobulin, a specific human protein involved in the HLA class I antigen complex. FIG. 25B: cells in these constructs stained positively for human PECAM1 and human anti-nuclei (FIG. 25B.1), and thus have properties of human endothelial cells while others stained positively for α-SMA and β₂-microglobulin (FIG. 25B.2) and thus have properties of human smooth muscle cells. FIG. 25C: the constructs with SML cells stained positively for α-SMA. Bar corresponds to 50 μm.

### DETAILED DESCRIPTION OF VARIOUS EMBODIMENTS

### Production of Vascular Cells from Stem Cells

In various embodiments, vascular progenitor cells are isolated from EBs using the hematopoietic/endothelial marker CD34. These vascular progenitor cells (i.e., CD34⁺ cells) are selectively induced to differentiate into either EL cells, SML cells, or HL cells. When implanted in nude mice, these cells contribute to the formation of functional microvessels containing mouse blood cells. In various embodiments, at least 5%, at least 7%, or at least 10% of the cells in the embryoid bodies are isolated for use with various embodiments of the inventions.

In various embodiments, cells characterized by expression of the endothelial/hematopoietic marker CD34 (CD34⁺ cells) are isolated from embryoid bodies (EBs) and cultured in EGM-2 medium supplemented with vascular endothelium growth factor (VEGF₁₆₅, 50 ng/mL). Cells cultured in this manner may be characterized by one or more of the following: a cobblestone cell morphology, expression of at least two or at least three of PECAM1, CD34, KDR/Flk-1, VE-CAD and vWF, the ability to take up acetylated low-density lipoprotein (ac-LDL), and/or formation of capillary-like structures when placed in Matrigel™, from Becton-Dickinson. Matrigel™ is further discussed below.

In various embodiments, CD34⁺ cells are cultured in EGM-2 medium supplemented with platelet-derived growth factor (PDGF_{BB}, 50 ng/mL). Cells cultured in this manner may be characterized by one or more of the following: a spindle-shape morphology, expression of at least one of α-SMA, SM-MHC, calponin, angiopoietin 1, caldesmon and SMα-22, and/or only sparse formation of capillary-like structures when placed in Matrigel. In various embodiments, fewer than 25%, fewer than 20%, fewer than 15%, fewer than 10%, or fewer than 5% of the cells produced according to this embodiment form capillary-like structures when placed in Matrigel.

In various embodiments, CD34⁺ cells are cultured on semisolid media with hematopoietic growth factors (e.g., 1% methylcellulose, 30% FBS, 1% BSA, 50 ng/ml stem cell factor, 20 ng/ml granulocyte-macrophage colony-stimulating factor, 20 ng/ml IL-3, 20 ng/ml IL-6, 20 ng/ml granulocyte colony-stimulating factor, and optionally 3 units/ml erythropoietin) (Kaufman et al, PNAS 2001, 98, 10716-10721).

In the absence of additional stimulus for further differentiation, these CD34⁺ cells are capable of generating large numbers of endothelial, smooth muscle and hematopoietic cells. In addition, vascular progenitor cells may be maintained in a viable state over long periods of time by cryopreservation according to any of the methods for conditioning, storage and thawing known to the skilled artisan.

### Vascularized Implants

In a various embodiments, the present inventions provide methods and structures where cells produced according to various embodiments of the present inventions are implanted into an animal to promote, e.g., the formation of vasculature. Accordingly, in various embodiments the present inventions provide implants for promoting vascularization and methods of vascularizing an implant.

In various embodiments, these cells may be combined with a cell support substrate including extracellular matrix components. The substrate may be a gel, for example, Matrigel™, from Becton-Dickinson. Matrigel™ is a solubilized basement membrane matrix extracted from the EHS mouse tumor (Kleinman, H. K., et al., Biochem. 25:312, 1986). The primary components of the matrix are believed to be laminin, collagen I, entactin, and heparan sulfate proteoglycan (perlecan) (Vukicevic, S., et al., Exp. Cell Res. 202:1, 1992). Matrigel™ is also believed to contain growth factors, matrix metalloproteinases (MMPs [collagenases]), and other proteinases (plasminogen activators [PAs]) (Mackay, A. R., et al., BioTechniques 15:1048, 1993). The matrix also is believed to include several undefined compounds (Kleinman, H. K., et al., Biochem. 25:312, 1986; McGuire, P. G. and Seeds, N. W., J. Cell. Biochem. 40:215, 1989), but it is believed that Matrigel does not contain any detectable levels of tissue inhibitors of metalloproteinases (TIMPs) (Mackay, A. R., et al., BioTechniques 15:1048, 1993).

In various embodiments, the gel may be a collagen I gel. Alternate gels that may be employed include hyaluronic acid, alginate, agarose, collagen, poly(ethylene glycol), poly(vinyl alcohol), dextran gels, fibrinogen, chitosan, self-assembling peptide gels and other non-cytophobic biocompatible gels. Examples of gels include those discussed in Lee & Mooney, Chemical Reviews 2001, 101(7), 1869-1879; Lutolf & Hubbell, Nature Biotechnology 2005, 23(1), 47-55; Williams et al., Tissue Engineering 2003, 9(4), 679-688; Anseth et al., Journal of controlled release 2002, 78, 199-209; Leach et al., Biotechnology and Bioengineering 2003, 82(5), 578-589; Lutolf et al. Advanced Materials 2003, 15(11), 888-892; Ferreira et al., Biomaterials 2002, 23(19, 3957-3967; Dikovsky et al., Biomaterials 2006, 27(8), 1496-1506; Kisiday et al., PNAS 2002, 99, 9996-10001; Silva et al., Science 2004, 303, 1352-1355; and Mwale et al., Tissue Engineering 2005, 11 (1-2), 130-140, all of which are incorporated herein by reference. Regardless of composition, the gel may also include other extracellular matrix components, such as glycosaminoglycans, fibrin, fibronectin, proteoglycans, and glycoproteins. The gel may also include basement membrane components such as collagen IV and laminin. Enzymes such as proteinases and collagenases may be added to the gel, as may cell response modifiers such as growth factors and chemotactic agents. Gels may also be modified to include cell adhesion epitopes or enzymatically degradable sequences.

The cells, either mixed with a gel or simply with a liquid carrier such as, e.g., PBS, may be injected directly into a tissue site where vasculogenesis is desired. For example, the cells may be injected into ischemic tissue in the heart or other muscle, where the cells will organize into tubules that will anastamose with existing cardiac vasculature to provide a blood supply to the diseased tissue. Other tissues may be vascularized in the same manner. The cells will incorporate into neovascularization sites in the ischemic tissue and accelerate vascular development and anastamosis. It is intended that in various embodiments the present inventions be used to vascularize all sorts of tissues, including connective tissue, muscle tissue, nerve tissue, and organ tissue. Non-blood duct networks may be found in many organs, such as the liver and pancreas, and the techniques of the invention may be used to engineer or promote healing in such tissues as well. For example, mixtures of EL and SML injected into the liver can develop into tubular networks around which native hepatocytes can develop other liver structures.

In various embodiments, CD34⁺ cells may be combined with a carrier (e.g., a gel or scaffold) in the same manner as more differentiated cells, e.g., EL and SML. In this embodiment, appropriate growth factors may be added to promote differentiation of the CD34⁺ cells into one or more of EL, SML, and HL such as those described in Ferreira et al., Biomaterials 2007, 28, 2706-2717. the contenst of which are incorporated herein by reference. The carrier may also include cell adhesion epitopes or extracellular matrix components that promote differentiation, such as those described in Shin, et al., Biomaterials, 2003, 24: 4353-4364, Yamashita, et al., Nature, 2000, 408: 92-96, Silva et al., Science 2004, 303, 1352-1355 the contents of all of which are incorporated herein by reference. This approach may originate other cell types than the vascular ones. For example, CD34⁺ cells isolated from peripheral blood originate cardiomyocytes, endothelial and smooth muscle cells. However, the population of vascular cells will still be enriched with respect to the original population.

Alternatively or in addition, CD34⁺ cells or one or more of SML, EL, or HL may be encapsulated in microparticles. Methods of forming hydrogel microcapsules are well known to those of skill in the art and are disclosed, for example, in U.S. Pat. No. 5,294,446, the entire contents of which are herein incorporated by reference. The hydrogels may be modified with cell adhesion epitopes and/or loaded with appropriate growth factors to promote differentiation in vivo (Mahoney & Saltzman, Nature Biotechnology 2001, 934-939).

Cells according to various embodiments of the present inventions may also be used to help heal cardiac vasculature following angioplasty. For example, a catheter can be used to deliver cells to the surface of a blood vessel following angioplasty or before insertion of a stent. The stent may be seeded with EL or other vascular progenitor cells. Blood vessels treated with adult endothelial cells exhibit accelerated re-endothelialization, preventing restenosis in the injured vessel (Parikh, et al. (2000) Advanced Drug Delivery Reviews, 42, 139-161). In various embodiments, cells may be seeded into a polymeric sheet and wrapped around the outside of a blood vessel that has undergone angioplasty or stent insertion (Nugent, et al. (2001) J. Surg. Res., 99, 228-234). The cells may also be mixed with a gel and infused into the polymer sheet instead of directly seeded onto the matrix. Cells according to various embodiments of the present inventions may also be used to seed vascular grafts (Nugent & Edelman, Circulation Research 2003, 92, 1068-1078).

If a stiffer implant is desired, the cells may be seeded onto a polymer matrix, for example, a sponge, which is then implanted into the desired tissue site. The cells may be mixed with a gel which is then absorbed onto the interior and exterior surfaces of the matrix and which may fill some of the pores of a spongy or other porous matrix. Capillary forces will retain the gel on the matrix before hardening, or the gel may be allowed to harden on the matrix to become more self-supporting.

The polymer matrix may serve simply as a delivery vehicle for the cells or may provide a structural or mechanical function. The matrix may be formed in any shape, for example, as particles, a sponge, tube, sphere, strand, coiled strand, capillary network, film, fiber, mesh, or sheet. The shape and size of the final implant may be adapted for the implant site and tissue type. Alternatively or in addition, the matrix may be formed with a microstructure similar to that of the extracellular matrix that is being replaced. Mechanical forces imposed on the matrix by the surrounding tissue will influence the cells on the artificial matrix and promote the regeneration of extracellular matrix with the proper microstructure. The mechanical properties of the matrix may also be optimized to mimic those of the tissue at the implant site. One skilled in the art will recognize how to adjust the molecular weight, tacticity, and cross-link density of the matrix material to control both the mechanical properties and, for degradable materials, the degradation rate.

The porosity of the matrix may be controlled by a variety of techniques known to those skilled in the art. The minimum pore size and degree of porosity is dictated by the need to provide enough room for the cells and for nutrients to filter through the matrix to the cells. The maximum pore size and porosity is partially indicated by the desired ability ability of the matrix to maintain its mechanical stability after seeding. As the porosity is increased, use of polymers having a higher modulus, addition of stiffer polymers as a co-polymer or mixture, or an increase in the cross-link density of the polymer may all be used to increase the stability of the matrix with respect to cellular contraction.

The matrices may be made by any of a variety of techniques known to those skilled in the art. Salt-leaching, porogens, solid-liquid phase separation (sometimes termed freeze-drying), and phase inversion fabrication may all be used to produce porous matrices. Fiber pulling and weaving (see, e.g. Vacanti, et al., (1988) Journal of Pediatric Surgery, 23: 3-9) may be used to produce matrices having more aligned polymer threads. Those skilled in the art will recognize that standard polymer processing techniques may be exploited to create polymer matrices having a variety of porosities and microstructures.

Preferably, the polymer matrix is biodegradable. Suitable biodegradable matrices are well known in the art and include collagen-GAG, collagen, fibrin, PLA, PGA, and PLA-PGA co-polymers. Additional biodegradable materials include poly(anhydrides), poly(hydroxy acids), poly(ortho esters), poly(propylfumerates), poly(caprolactones), polyamides, polyamino acids, polyacetals, biodegradable polycyanoacrylates, biodegradable polyurethanes, poly(glycerol sebacates), especially elastomeric poly(glycerol sebacates), and polysaccharides. Non-biodegradable polymers may also be used as well. Other non-biodegradable, yet biocompatible polymers include polypyrrole, polyanilines, polythiophene, polystyrene, polyesters, non-biodegradable polyurethanes, polyureas, poly(ethylene vinyl acetate), polypropylene, polymethacrylate, polyethylene, polycarbonates, and poly(ethylene oxide). Those skilled in the art will recognize that this is not a comprehensive, list of polymers appropriate for tissue engineering applications.

PLA, PGA and PLA/PGA copolymers are particularly useful for forming the biodegradable matrices. PLA polymers are usually prepared from the cyclic esters of lactic acids. Both L(+) and D(−) forms of lactic acid can be used to prepare the PLA polymers, as well as the optically inactive DL-lactic acid mixture of D(−) and L(+) lactic acids. PGA is the homopolymer of glycolic acid (hydroxyacetic acid). In the conversion of glycolic acid to poly(glycolic acid), glycolic acid is initially reacted with itself to form the cyclic ester glycolide, which in the presence of heat and a catalyst is converted to a high molecular weight linear-chain polymer. The erosion of the polyester matrix is related to the molecular weights. The higher molecular weights, weight average molecular weights of 90,000 or higher, result in polymer matrices which retain their structural integrity for longer periods of time; while lower molecular weights, weight average molecular weights of 30,000 or less, exhibit shorter matrix lives. The tacticity of the polymer also influences the modulus. Poly(L-lactic acid) (PLLA) is isotactic, increasing the crystallinity of the polymer and the modulus of mixtures containing it. One skilled in the art will recognize that the molecular weight and crystallinity of any of the polymers discussed above may be optimized to control the stiffness of the matrix. Likewise, the proportion of polymers in a co-polymer or mixture may be adjusted to achieve a desired stiffness.

Another polymer that may find particular use in certain embodiments is elastomeric poly(glycerol sebacate). This polymer may be produced by producing a branched glycerol-sebacate prepolymer that is then cross-linked to produce an elastomer. The reaction conditions (e.g., time and temperature), may be adjusted to control the extent of the cross-linking reaction and therefore the elastic modulus and degradation rate of the material. Catalysts may be employed to regulate the molecular weight and the degree of cross-linking as understood by those of skill in the art. The resulting polymer is tough and biodegradable and may be functionalized with growth factors or other materials via the hydroxyl groups on the glycerol.

Co-polymers, mixtures, and adducts of the above polymers may also be used in the practice of the invention. Indeed, co-polymers may be particularly useful for optimizing the mechanical and chemical properties of the matrix. For example, a polymer with a high affinity for stem cells may be combined with a stiffer polymer to produce a matrix having the requisite stiffness to resist collapse. For example, PLA may be combined with poly(caprolactone) or PLGA to form a mixture. Both the choice of polymer and the ratio of polymers in a co-polymer may be adjusted to optimize the stiffness of the matrix.

In various embodiments, a cell response modifier such as a growth factor or a chemotactic agent may be added to the polymer matrix. Such a modifier, for example, vascular endothelial-derived growth factor or PDGFBB, may be used to promote differentiation of the vascular progenitor cells. Numerous growth factors have been implicated in the complex processes of vasculogenesis, angiogenesis and hematopoietic differentiation (for reviews, see Carmeliet et al., Nature Medicine 2000, 6, 389-395; and Yancopoulos et al., Nature 2000, 407, 242-248). Although some (i.e. VEGF and PDGF) are more dominant in their effects than others, effective differentiation of progenitor cells into differentiated cells is typically a result of the combined, and temporally coordinated action of a number of factors. Other growth factors that may enhance the differentiation of vascular progenitor cells are: fibroblast growth factor (FGF), granulocyte-macrophage colony stimulating factor (GM-SCF), angiopoietin (Ang), ephrin (Eph), placental growth factor (PIGF), tumor growth factor, transforming growth factor 13-1 [ (TGF)-β1], cytokines, erythropoietin, thrombopoietin, transferrin, insulin, stem cell factor (SCF), granulocyte colony-stimulating factor (G-CSF) retinoic acid and granulocyte-macrophage colony stimulating factor (GM-CSF), among others.

The modifier may be selected to recruit cells to the matrix or to promote or inhibit specific metabolic activities of cells recruited to the matrix. Examples of growth factors include epidermal growth factor, bone morphogenetic protein, TGFβ, hepatocyte growth factor, platelet-derived growth factor, TGFα, IGF-I and II, hematopoetic growth factors, heparin binding growth factor, peptide growth factors, and basic and acidic fibroblast growth factors. In various embodiments growth factors such as nerve growth factor (NGF) or muscle morphogenic factor (MMP) may be desirable. The particular growth factor employed should be appropriate to the desired cell activity.

Bioactive agents, biomolecules, and small molecules may also be added to the polymer matrix or to a culture medium before seeding. For example, addition of fibronectin, integrins, or oligonucleotides that promote cell adhesion, such as RGD, may be added to the polymer matrix. Chemotactic or anti-inflammatory agents may be added to the matrix to influence the behavior of cells in the tissue surrounding an implanted matrix.

The cell-seeded polymer matrix, with or without the gel, may be implanted into any tissue, including connective, muscle, nerve, and organ tissues. For example, an implant placed into a bony defect will attract cells from the surrounding bone which will synthesize extracellular matrix, while the vascular progentor cells promote formation of blood vessels. The blood supply for the new bone will be provided as the new ECM is formed and mineralized. An implant placed into a skin defect will promote dermis formation and provide a vascular network to supply nutrients to the newly formed skin.

The cells may be seeded onto a tubular substrate. For example, the polymer matrix may be formed into a tube or network. Such tubes may be formed of natural or synthetic ECM materials such as PLA or collagen or may come from natural sources, for example, decellularized tubular grafts. The vascular progenitor cells will coat the inside of the tube, forming an artificial channel that can be used for a heart bypass. In various embodiments, use of vascular progenitor cells of the present inventions may reduce thrombosis post-implantation.

The cells may be allowed to proliferate on the polymer matrix or tubular substrate before being implanted in an animal. During proliferation, mechanical forces may be imposed on the implant to stimulate particular cell responses or to simulate the mechanical forces the implant will experience in the animal. For example, a medium may be circulated through a tubular substrate in a pulsatile manner (i.e., a hoop stress) or with sufficient speed to exert a sheer stress on cells coating the inside of the tube. A hydrostatic force or compressive force may be imparted on an implant that will be deposited within an organ such as the liver, or a tensile stress may be imparted on an implant that will be used in a tissue that experiences tensile forces.

Cells that are recruited to the implant may also differentiate into other cell types. Bone cell precursors migrating into a bone implant can differentiate into osteoblasts. Mesenchymal stem cells migrating into a blood vessel can differentiate into muscle cells. Endothelial cells forming tubular networks in liver can induce the formation of liver tissue.

In various embodiments, the vascular progenitor cells are mixed with another cell type before implantation. The cell mixture may be suspended in a carrier such as a culture medium or in a gel as described above. The cells may be co-seeded onto a polymer matrix or combined with a gel that is absorbed into the matrix. While cumbersome, it may be desirable to seed one cell type directly onto the matrix and add the second cell type via a gel. Any ratio of vascular progenitor cells to the other cell type or types may be used. One skilled in the art will recognize that this ratio may be easily optimized for a particular application. Examples of ratios of vascular progenitor cells to other cells are at least 10% (e.g., 1:9), at least 25%, at least 50% (e.g., 1:1), at least 75%, and at least 90%. Smaller ratios, for example, less than 10%, may also be employed.

Any cell type, including connective tissue cells, nerve cells, muscle cells, organ cells, or other stem cells, may be combined with the vascular progenitor cells. For example, osteoblasts may be combined with the vascular progenitor cells to promote the co-production of bone and its vasculature in a large defect. Fibroblasts combined with vascular progenitor cells and inserted into skin will produce fully vascularized dermis. Other examples of cells that may be combined with the vascular progenitor cells of the invention include ligament cells, lung cells, epithelial cells, smooth muscle cells, cardiac muscle cells, skeletal muscle cells, islet cells, nerve cells, hepatocytes, kidney cells, bladder cells, and bone-forming cells.

Furthermore, the mechanical interactions of cells and their extracellular matrix influence cellular processes. To further promote differentiation along a desired path, exogenous mechanical forces may be used as a cell response modifier to mimic the mechanical forces exerted by tissues. For example, endothelial cells are exposed to shear forces as blood flows through arteries and veins. Muscle is exposed to both uniform and non-uniform tensile stresses. Organ tissues are exposed to hydrostatic stresses and other compressive stresses. Imposition of mechanical forces on cell-seeded matrices in vitro will influence the production of actin by the seeded stem cells, in turn influencing the degree and type of metabolic activity of the cells and the microstructure of the extracellular matrix they produce.

Similarly, electrical stimulation may be used to influence cell differentiation and metabolism. For example, bone is piezoelectric, and muscle contracts and relaxes in response to electrical signals conducted through nerves. In vitro electrical stimulation imitating the electrical activity of the desired tissue may cause ES cells seeded on a three-dimensional matrix to produce tissue having the electrical characteristics of that tissue.

The shape and microstructure of the polymer matrix and the exogenous forces imposed on the seeded polymer may be optimized for a specific tissue. For example, a medium may be circulated through a seeded tubular substrate in a pulsatile manner (i.e., a hoop stress) to simulate the forces imposed on an artery, or the medium may be used to exert a shear stress on stem cells lining the inside of a tube (Niklason, et al., (1999) Science 284, 489-93; Kaushall, et al., (2001) Nat. Med., 7, 1035-1040). The polymer strands in the matrix may be aligned to mimic the tissue structure of muscle, tendon, or ligament or formed into tubular networks to promote the formation of vasculature.

Even before seeded ES cells are fully differentiated, they can organize themselves into three-dimensional structures characteristic of almost all animal tissue after being exposed to a cell response modifier. Seeded on matrices that can provide a physiologic response to mechanical forces exerted by the stem cells, the stem cells will be able to differentiate and develop under conditions that are more similar to a physiologic environment than a two dimensional petri dish. Indeed, integration of the implant into a tissue site may proceed more quickly or efficiently before the ES cells are terminally differentiated.

### Vascular Models

Differentiating cultures or vascular tissues prepared from vascular progenitor cells of the present invention also provide a model suitable for the investigation of processes affecting vascular development and function. For example, in various embodiments, the cells and tissues of the present inventions may be cultured in the presence of suspected toxic materials, antibodies, teratogens, drugs , or exposed to non-standard environmental factors such as temperature, gas partial pressure and pH, or co-cultured in the presence of cells from other tissues or other organisms. Changes in parameters of growth and development, such as failure or delay of endothelial marker expression, loss of proliferative capacity, or disorganization of in vitro vascularization can be assessed to determine the effect of various factors.

### EXAMPLES

Various aspects and embodiments of the present inventions may be further understood in light of the following examples, which are not exhaustive and which should not be construed as limiting the scope of the present inventions in any way.

### Example 1

### Cell Culture, Differentiation and Implantation

This example presents data on the culturing of hESCs, isolation of vascular progenitor cells and different of the progenitor cells into EL cells or SML cells. In addition, implantation studies in nude mice of a Matrigel matrix comprising differentiated cells of produce according to this example are also presented. In addition to the text below, the brief descriptions of the Figures also contains information regarding this example.

### Materials and Methods

Cell culture. Human ESC lines H9 and H13 were grown (passages 25 to 45; WiCell, Wisconsin) on an inactivated mouse embryonic feeder layer (MEF, Cell Essential, Boston, Mass.), substantially as described by M. Amit et al. in “Clonally derived human embryonic stem cell lines maintain pluripotency and proliferative potential for prolonged periods of culture” Dev Biol., 227, pp. 271-8 (2000). All the studies were performed with H9 cell line unless stated otherwise. To induce the formation of human EBs, the undifferentiated hESCs were treated with 2 mg/mL type IV collagenase (Invitrogen) for 2 h, and then transferred (2:1) to low attachment plates (Ø=10 cm, Ref: 3262, Corning) containing 10 mL of differentiation medium [80% Knockout-Dulbecco's Modified Eagle Medium (Invitrogen), 20% Knockout-serum (KO-SR, Invitrogen) or fetal bovine serum (FBS, Hyclone), 1 mM L-glutamine, 0.1 mM β-mercaptoethanol and 1% nonessential amino acid stock (all from Invitrogen)]. EBs were cultured for 12 days at 37° C., and 5% CO₂ in a humidified atmosphere, with changes of media every 3-4 days. To serve as controls, human vascular smooth muscle cells (hVSMCs) and human umbilical vein endothelial cells (HUVECs) were obtained from Cambrex and cultured in EGM-2 or SmGM-2 media (Cambrex). Medium was changed every other day.

Isolation and culture of CD34⁺ cells. Selection of CD34⁺ cells at day 10 was performed by labeling the hES cells with the anti-CD34 antibody (QBEND/10, Miltenyi Biotec) conjugated with magnetic beads. The magnetically labelled cells were separated into CD34⁺ and CD34⁻ populations using a LS-MACS column (Miltenyi Biotec). CD34 enrichment was confirmed by flow cytometry analysis using a different anti-CD34 antibody (AC136, Miltenyi Biotec). Isolated CD34⁺ cells were grown on 24-well plates (30,000 cells/well) coated with 1% gelatin and containing EGM-2 medium, EGM-2 medium supplemented with VEGF₁₆₅ (50 ng/mL, R&D Systems), or PDGF_{BB} (50 ng/mL, R&D Systems).

Transplantation into nude mice. EL and SML cells alone (3^{rd} passage, 0.5×10⁶ cells in ca. 20 μL of EGM-2 media), or EL cells mixed with SML cells (3:1; 0.5×10⁶ cells in total, in 20 μL of EGM-2 media) were suspended in 0.350 mL of Matrigel (BD Biosciences), on ice. The cell suspension was injected subcutaneously (23-gauge needle) in each side of the dorsal region of 4-week-old male balb/c nude mice (2 implants per mice; 3 mice per experimental condition). Matrigel without cells was used as control. After 28 days, the implants were removed from mice after euthanasia by CO₂ asphyxiation, fixed overnight in 10% (v/v) buffered formalin at 4° C., embedded in paraffin, and sectioned for histological examination.

Immunostaining. For staining, EBs were transferred to gelatin-coated cover slips with differentiation medium containing 20% (v/v) FBS. After attachment to the cover slips (overnight), the EBs were fixed with 4% (w/v) paraformaldehyde for 30 minutes at room temperature. For the evaluation of SMC or EC phenotypes in CD34⁺ cells a similar fixation procedure was adopted. After blocking with 3% BSA solution, the cells were stained for 1 h with the following anti-human primary antibodies: PECAM1 (JC70A), CD34 (QBEnd 10), vWF (F8/86), α-SMA (1A4), SM-MHC (SMMS-1), calponin (CALP) (all from Dako) and VE-cad (F-8; Santa Cruz Biochemicals). In each immunofluorescence experiment, an isotype-matched IgG control was used. Binding of primary antibodies to specific cells was detected with anti-mouse IgG Cy3 conjugate (Sigma). Cell nuclei were stained with 4′,6-diamidino-2-phenylindole (DAPI) or Topro-3 (Sigma) Immunostaining was examined with either a fluorescence microscope (Nikon) or Zeiss LSM 510 confocal microscope.

For uptake of Dill-labelled acetylated low-density lipoprotein (ac-LDL), differentiated CD34⁺ cells were incubated with 10 mg/mL Dill-labelled ac-LDL (Biomedical Technologies) for 4 h at 37° C. After incubation, cells were washed three times with PBS, fixed with 4% (w/v) paraformaldehyde for 30 min, and visualized with a fluorescent microscope.

Immunohistochemical staining of explants from animal study was carried out using the Dako EnVision™+/HRP kit (Dako), with prior heat treatment at 95° C. for 20 min in ReVeal buffer (Biocare Medical), or trypsin (1 mg/mL), for epitope recovery. For immunofluorescent staining, anti-mouse IgG Cy3 conjugate was used as secondary antibody followed by DAPI nuclear staining. The primary antibodies were anti-human PECAM1 (1:20), anti-human collagen type IV (1:500, Sigma), biotinylated Ulex europaeus agglutinin-1 (UEA-1, 1:100, Vector Laboratories), anti-α-SMA (1:50), and the corresponding isotype controls. The number of microvessels that were immunoreactive for human collagen type IV was counted in 7 random fields from at least four implants (2 sections for each implant) at ×20 magnifications (corresponding to an area of 3.4×10⁵ μm²).

Fluorescence-activated cell sorting (FACS) analysis. Undifferentiated hES, HUVEC/hVSMC or CD34⁺ cells grown in different growth media were dissociated with non-enzymatic cell dissociation solution (Sigma) for 10 min. EBs were dissociated with 0.4 U/mL collagenase B (Roche Diagnostics) for 2 h in a 37° C. incubator, followed by treatment with cell dissociation solution for 10 min, followed by gentle pipetting. Single cells were aliquoted (1.25−2.5×10⁵ cells were used per condition) and stained with either isotype controls or antigen-specific antibodies: SSEA-4-PE (MC813-70, R&D Systems), PECAM1-FITC (30884X, BD Pharmingen), CD34-PE/CD34-FITC (AC136, Miltenyi Biotec), KDR/Flk 1-PE (89106, R&D) and CD45-FITC (HI30, BD Pharmingen). Cells were analysed without fixation on a FACScan (Becton Dickinson), using propidium iodide to exclude dead cells. For α-SMA, SM-MHC (all from Dako) and alkaline phosphatase-APC (R&D systems) markers, an intrastain kit (Dako) was used for the fixation and permeabilization of cell suspensions. In case of α-SMA and SM-MHC, the monoclonal antibodies were conjugated with a FITC-secondary antibody (Dako). Data analysis was carried out using CellQuest software.

Reverse Transcription-Polymerase Chain Reaction (RT-PCR) analysis. Total RNA was extracted using trizol (Invitrogen) according to manufacturer's instructions. Total RNA was quantified by a UV spectrophotometer, and 1 μg was used for each RT sample. RNA was reversed transcripted with M-MLV and oligo (dT) primers (Promega) according to manufacturer's instructions. PCRs were done with BIOTAQ DNA Polymerase (Bioline) using 1 μL of RT product per reaction. To ensure semi-quantitative results of the RT-PCR assays, the number of PCR cycles for each set of primers was verified to be in the linear range of the amplification. In addition, all RNA samples were adjusted to yield equal amplification of glyceraldehyde-3-phosphate dehydrogenase (GAPDH) as an internal standard.

Table 1 presents information on primer sequences, various reaction conditions and optimal cycle numbers used for the RT-PCR analyses of vascular markers for various gene transcripts in this example. For the data of Table 1, the PCR conditions comprised the following: 5 minutes at 94° C. (hot start), 30 to 40 cycles (actual number noted in the table); 94° C. for 30 seconds, annealing temperature (noted in the table) for 30 seconds; 72° C. for 30 seconds. A final 7 minutes extension at 72° C. was performed at the end. The amplified products were separated on 2% agarose gels with ethidium bromide.

TABLE 1

| | | | | Annealing | |
|---|---|---|---|---|---|
| | | Product | | Temp. | [MgCl₂] |
| Gene transcript | Primer sequences (5′ to 3′, F_{w}/Rᵥ) | (bp) | Cycles | (° C.) | (mM) |
| Angiopoietin-1 | GGGGGAGGTTGGACTGTAAT | 362 | 35 | 60 | 1.5 |
| | AGGGCACATTTGCACATACA | | | | |
| Angiopoietin-2 | GGATCTGGGGAGAGAGGAAC | 535 | 35 | 60 | 1.5 |
| | CTCTGCACCGAGTCATCGTA | | | | |
| Tie2 | ATCCCATTTGCAAAGCTTCTGGCTGGC | 512 | 35 | 60 | 1.5 |
| | TGTGAAGCGTCTCACAGGTCCAGGATG | | | | |
| VE-cad | ACGGGATGACCAAGTACAGC | 596 | 35 | 60 | 1.5 |
| | ACACACTTTGGGCTGGTAGG | | | | |
| Von | ATGTTGTGGGAGATGTTTGC | 656 | 40 | 55 | 1.0 |
| Willebrand | GCAGATAAGAGCTCAGCCTT | | | | |
| Factor (vWF) | | | | | |
| Caldesmon | AACAACCTGAAAGCCAGGAGG | 530 | 35 | 60 | 1.5 |
| | GCTGCTTGTTACGTTTCTGC | | | | |
| SMα-22 | CGCGAAGTGCAGTCCAAAATCG | 928 | 35 | 60 | 1.5 |
| | GGGCTGGTTCTTCTTCAATGGGG | | | | |
| GAPDH | AGCCACATCGCTCAGACACC | 302 | 27 | 60 | 1.5 |
| | GTACTCAGCGCCAGCATCG | | | | |

Matrigel assay. For Matrigel differentiation assay, a 24-well plate was coated with 0.4 mL of Matrigel per well and incubated for 30 minutes at 37° C. CD34⁺ cells differentiated for 3 passages in EGM-2 medium or EGM-2 medium supplemented with VEGF₁₆₅ or PDGF_{BB} were seeded on top of the Matrigel at a concentration of 2.5×10⁴−1×10⁵ per 300 μL of culture medium. After 1 h of incubation at 37° C., 1 mL of medium was added. Cord formation was evaluated by contrast-phase microscopy 24 h after seeding the cells.

Electron Microscopy. Cells seeded in Matrigel-coated 24-well plate were fixed for 1 h in 2.5% (w/v) glutaraldehyde, 3% (w/v) paraformaldehyde, and 5.0% (w/v) sucrose in 0.1 M sodium cacodylate buffer (pH 7.4) and then post-fixed in 1% (w/v) OsO₄ in veronal-acetate buffer for 1 h. The cells were stained en bloc overnight with 0.5% uranyl acetate in veronal-acetate buffer (pH 6.0), dehydrated, and embedded in Spurrs resin. Sections were cut on a Reichert Ultracut E at a thickness of 70 nm with a diamond knife. Sections were examined with a Philips EM410 electron microscope.

Statistical analysis. Statistical significance was determined using an unpaired Student t test. In this example results were considered statistically significant when P 0.05.

### Results

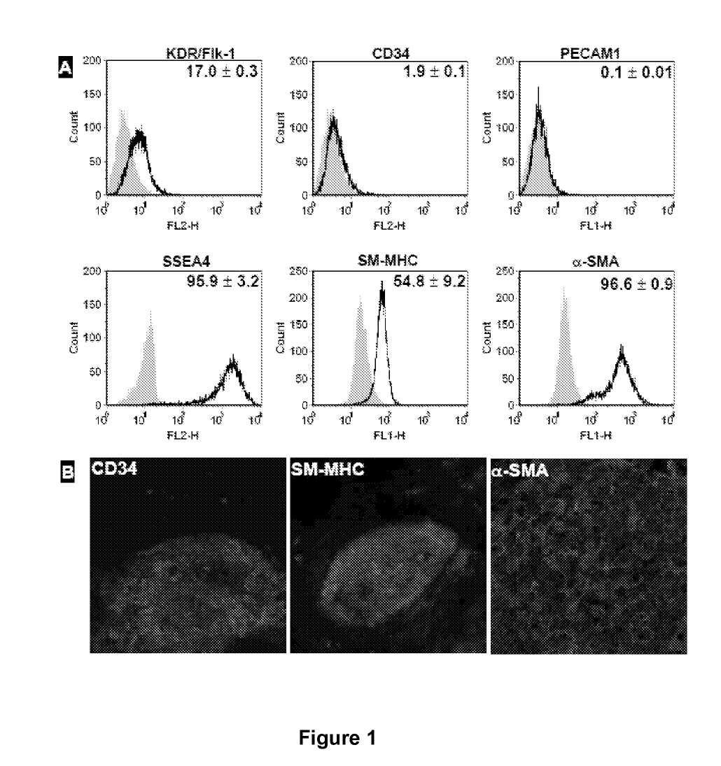

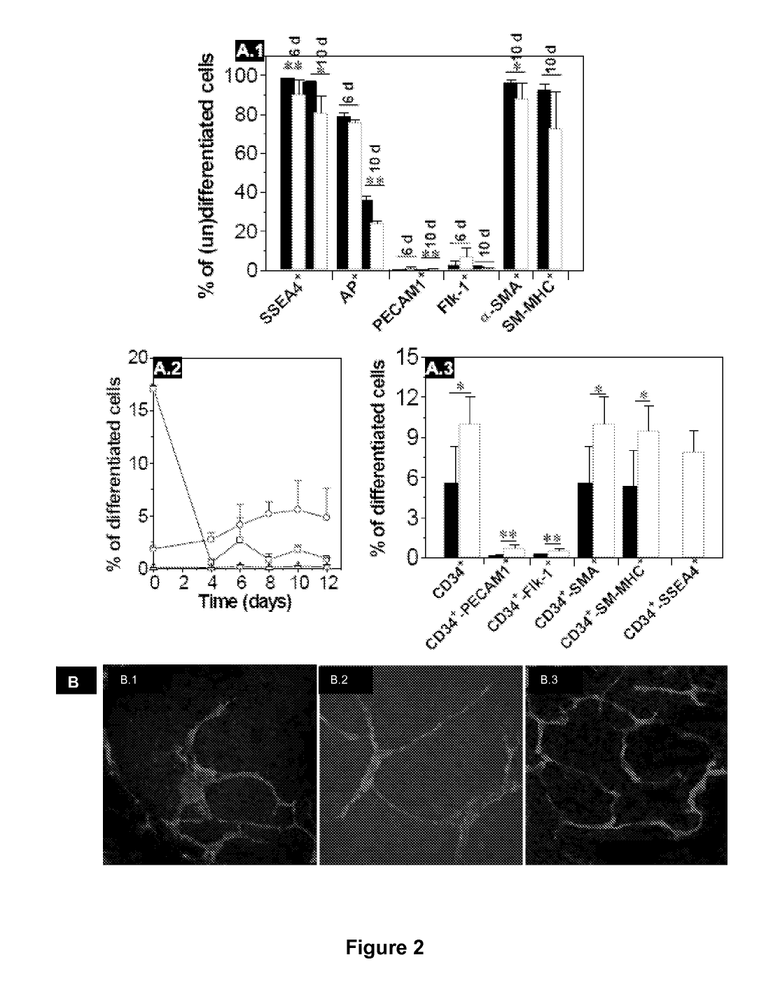

Vascular differentiation during EB development: effects of serum supplements. EBs were grown in medium containing KO-SR or FBS and analysed over a two week period for expression of well-characterized EC (PECAM1, CD34 and KDR/Flk-1), SMC (α-SMA and SM-MHC), and undifferentiated embryonic stem cell markers [SSEA4 and alkaline phosphatase (AP)] using FACS and immunocytochemistry. Initial hESCs expressed low or undetectable levels of EC markers such as CD34 and PECAM1, significant levels of KDR/Flk-1 (FIGS. 1A and 1B) as well as high levels of α-SMA and SM-MHC (FIGS. 1A and 1B). Thus, some of the EC and SM cell markers are already expressed in the undifferentiated hESC. The removal of undifferentiated hESCs from MEFs, and subsequent culture as EBs in differentiation medium containing KO-SR, reduced the expression of AP and SSEA4 over time, indicating that cells were undergoing differentiation (FIG. 2A.1). During this differentiation process, α-SMA and SM-MHC were highly expressed for 10 days (FIG. 2A.1), expression of CD34 marker peaked around day 10, and PECAM1 and KDR/Flk-1 expression levels remained low after 12 days of differentiation (FIG. 2A.2). At day 10, CD34⁺ cells co-expressed low levels of PECAM1 (˜3% of CD34⁺ cells) and KDR/Flk-1 (˜5%) but high levels of α-SMA (˜100%) and SM-MHC (˜100%) (FIG. 2A.3).

The effect of serum supplementation on EB differentiation was also evaluated in this example. Use of FBS instead of KO-SR resulted in a slightly accelerated differentiation process, as indicated by the decrease of AP and SSEA4 levels (FIG. 2A.1), and a significant (P<0.05) increase in the expression of CD34 (FIG. 2A.3). At day 10, CD34⁺ cells co-expressed significantly (P<0.01) higher levels of PECAM1 (˜7%) and KDR/Flk-1 (˜6%) than cells from EBs grown in medium containing KO-SR (FIG. 2A.3). At this time, the CD34⁺ cells also co-expressed high levels of SSEA4 (˜79%), α-SMA (˜100%), SM-MHC (˜95%) and minimal levels of the hematopoietic marker CD45 (˜3%, data not shown). EBs grown in medium with FBS showed lower expression of α-SMA and SM-MHC than EBs grown in medium containing KO-SR (FIG. 2A.1). Taken together, medium supplementation with FBS enhanced the vascular differentiation of cells in EBs and contributed to high yields of CD34⁺ cells. Furthermore, CD34⁺ cells co-expressed low levels of other endothelial markers, and high levels of SMC and undifferentiated stem cell markers.

Formation of vessel-like structures in EBs. Confocal analysis of EBs cultured for 10 days showed that CD34⁺ cells formed extensive vascular networks (FIGS. 2B.1 and 2B.2). This process was more evident in EBs grown in medium containing FBS than KO-SR. The vessel-like structures resemble ones we previously observed in PECAM1⁺ cells (FIG. 2B.3) (Levenberg S, Golub J S, Amit M, Itskovitz-Eldor J, Langer R. Endothelial cells derived from human embryonic stem cells. Proc Natl Acad Sci USA. 2002; 99:4391-6.); however, these structures were more frequent for CD34⁺ than for PECAM⁺ cells. As confirmed by FACS analysis (FIGS. 2A.1 and 2A.3), all PECAM1⁺ cells co-expressed CD34.

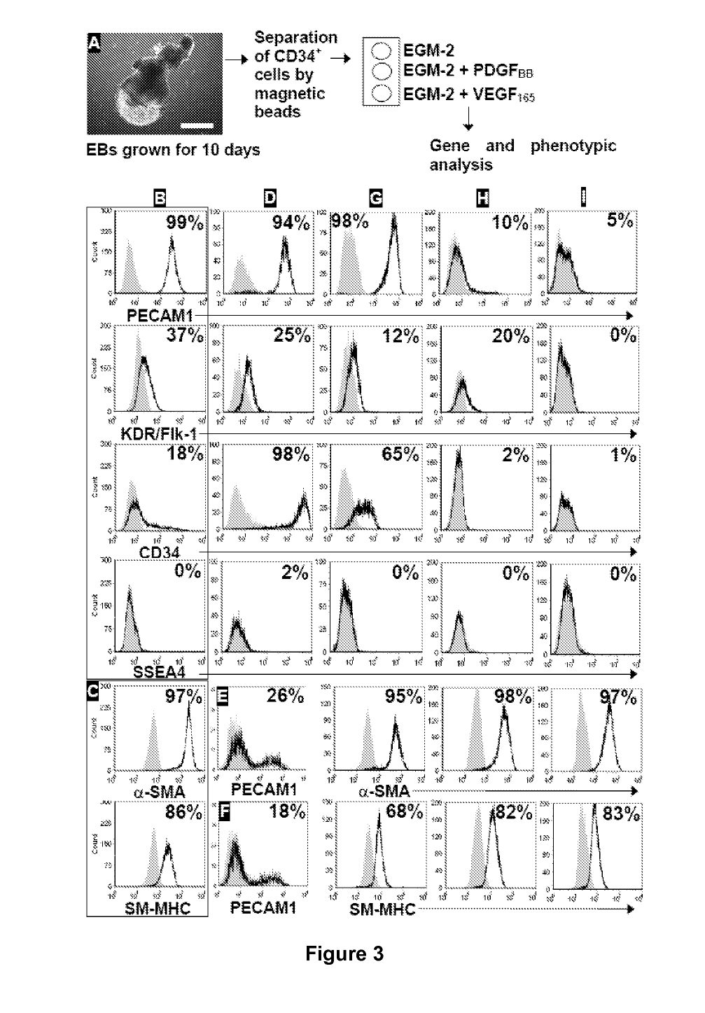

Induction of CD34⁺ cell differentiation into endothelial and smooth muscle cell lineages. CD34 marker was used to isolate vascular progenitor cells by magnetic selection from EBs grown in differentiation medium with FBS, for 10 days (FIG. 3A). These conditions were selected in this example because of high expression of CD34 during EB development (FIG. 2A.2). The cells isolated were 62% pure for CD34 antigen (approximately a six fold enrichment of the initial population of cells). The isolated CD34⁺ cells were cultured with EGM-2 medium alone or medium supplemented with VEGF₁₆₅ (50 ng/mL) or PDGF_{BB} (50 ng/mL) (FIG. 3A), since VEGF₁₆₅ and PDGF_{BB} have been reported to facilitate the differentiation of stem cells into ECs and SMCs, respectively.

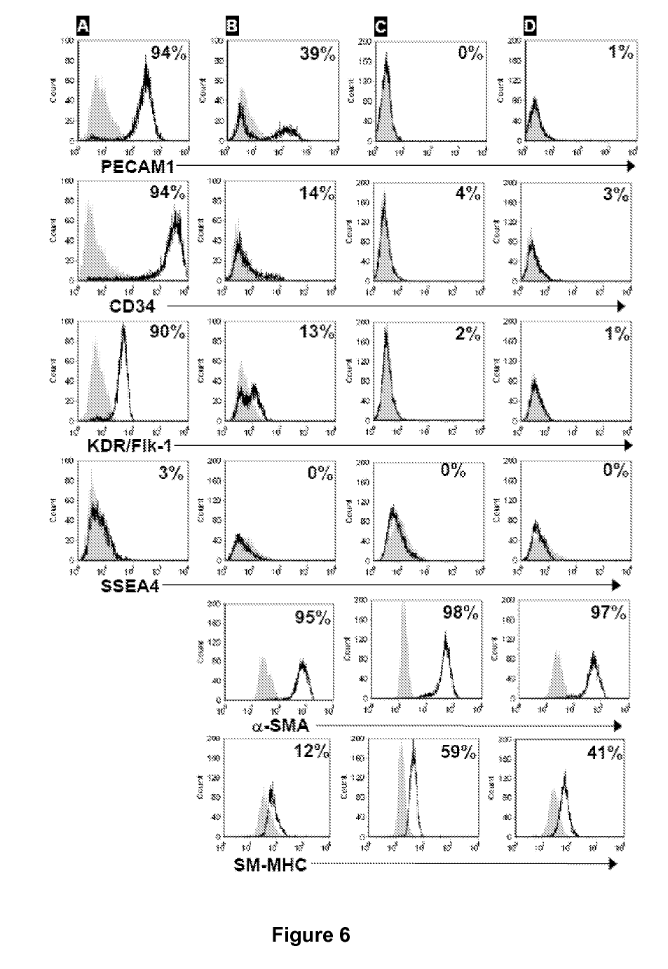

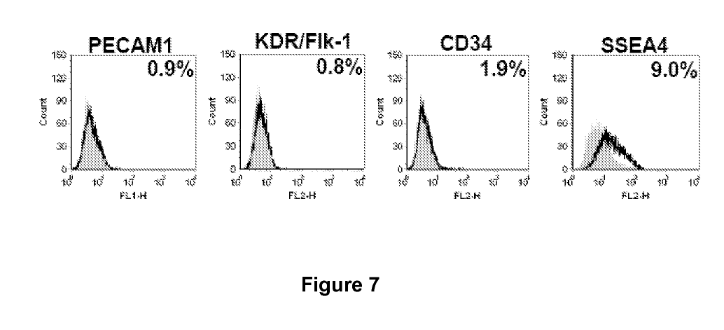

CD34⁺ cells cultured in VEGF-supplemented EGM-2 medium for 1 passage (10-15 days after cell seeding) expressed high levels of endothelial cell markers (FIG. 3D). Similar results were obtained with H13 cell line (FIG. 6). As compared to human umbilical vein endothelial cells (HUVECs), CD34⁺ cells had slightly lower expression of PECAMI and KDR/Flk1 (FIG. 3B), and higher expression of CD34. At this stage, the cells lost almost completely the marker SSEA4 indicative of their differentiation state. CD34⁻ cells grown in the same conditions as CD34⁺ cells showed minimal expression of the endothelial markers (FIG. 7), indicating that CD34⁺ cells, but not the CD34⁻ cells can be effectively induced to endothelial lineage. CD34⁺ cells cultured in EGM-2 medium (FIG. 3E) or EGM-2 medium supplemented with PDGF_{BB} (FIG. 3F) for 1 passage showed a much lower expression of PECAM1 (26% and 18%, respectively) than the CD34⁺ cells cultured in VEGF-supplemented medium (94%). As EGM-2 medium contains <5 ng/ml VEGF₁₆₅ (as measured by an ELISA kit), VEGF concentration has an effect on the endothelial differentiation of CD34⁺ cells.

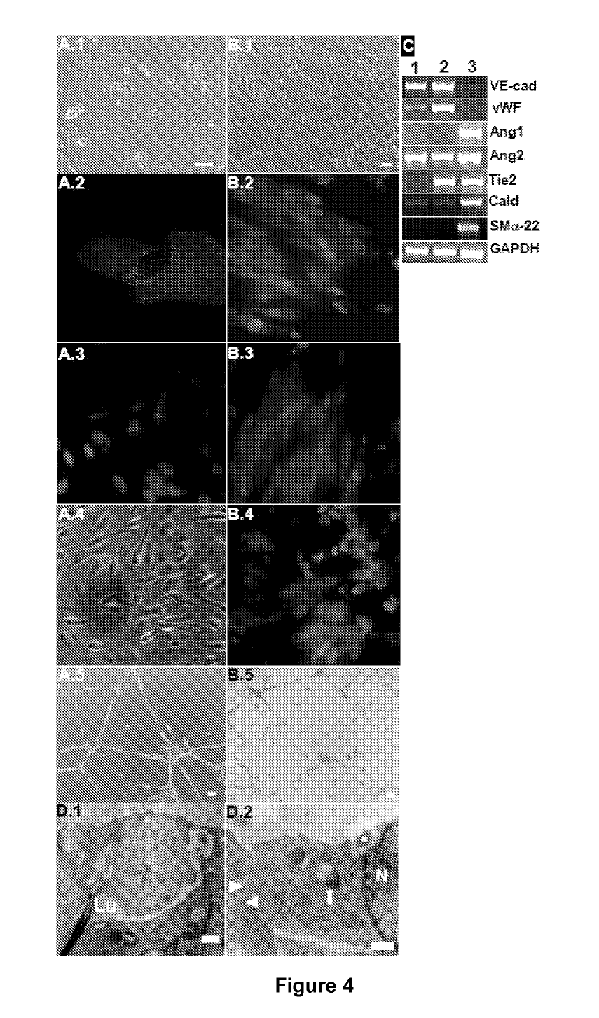

The proliferation rate of CD34⁺ cells cultured in VEGF-supplemented medium was high, achieving 20 population doublings over a two-month period (data not shown). FACS analyses of CD34⁺ cells cultured for 3 passages (FIG. 3G) showed the expression of PECAM1 was comparable to that in HUVEC (FIG. 3B) but different regarding the expression of CD34 and KDR/Flk-1 markers. CD34⁺ cells isolated from H13 cell line and differentiated in VEGF-supplemented medium presented lower levels of PECAM1 and CD34 compared to the H9 cell line (FIG. 6), suggesting slightly different differentiation profiles in the two cell lines. These cells displayed high expression of α-SMA and SM-MHC; however, as confirmed by immunocytochemistry, the cytoplasmic staining was diffused, indicating atypical actin and myosin organisation (data not shown). Cells stained positively for VE-cadherin at cell-cell adherent junctions, produced vWF cytoplasmitically, and were able to incorporate ac-LDL (FIG. 4A). Analysis by RT-PCR demonstrated that these cells express other common markers of vascular cells, including angiopoietin 2, a soluble ligand expressed by endothelial cells, and Tie2 receptor, but are negative for SMC markers including SMα-22 and angiopoietin1 (FIG. 4C).

Cells cultured in EGM-2 medium or PDGF_{BB}-supplemented medium for 3 passages expressed high levels of α-SMA, SM-MHC and calponin (FIGS. 3 and 4), low levels of endothelial markers (≦20%), and no detectable expression of the undifferentiating stem cell marker SSEA4. The levels of SMC markers were comparable to those observed in human vascular smooth muscle cells (hVSMC) (FIG. 3C). As confirmed by RT-PCR (FIG. 4C), PDGF_{BB}-supplemented EGM-2 medium upregulated the expression of definitive SMC markers, including caldesmon and SMα-22, and the expression of angiopoietin 1, a ligand produced by SMCs that activate the receptor Tie-2 found in ECs. This indicates that the presence of PDGF_{BB} contributed to cell maturation towards SMC phenotype. In addition, CD34⁺ cells grown in the presence of PDGF had higher proliferation rates than CD34⁺ cells grown in the presence of VEGF, with 42 population doublings over a two month period.

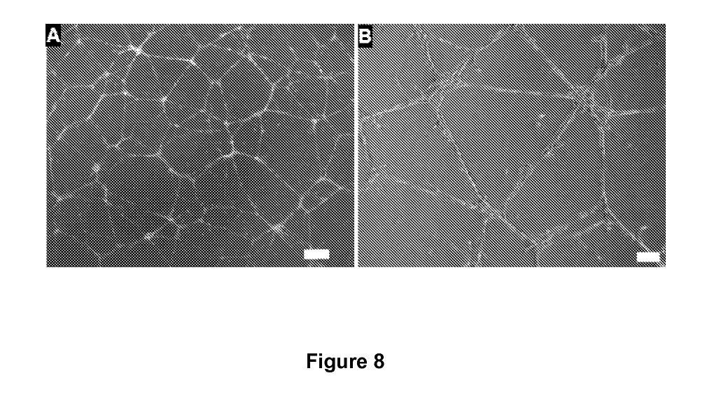

The ability of CD34⁺ cells, differentiated in VEGF or PDGF-supplemented medium, to form cord-like structures was also assessed by culturing these cells in the extracellular matrix basement membrane Matrigel. The CD34⁺ cells differentiated in VEGF-supplemented medium were able to spontaneously reorganize into cord-like structures when maintained in culture for 24 h (FIG. 4A.5 and FIG. 8). In contrast, CD34⁺ cells differentiated in EGM-2 medium containing PDGF_{BB} rarely formed cord-like structures (FIG. 4B.5). Electron micrographs of cord-sections formed by CD34⁺ cells differentiated in VEGF₁₆₅-supplemented medium showed the presence of a lumen (FIG. 4D.1), thus confirming the capacity of these cells to form vascular networks in vitro. In addition, these cells presented typical endothelial features, such as the presence of round or rod-shaped structures that resemble Weibel-Palade bodies and tight junctions between cells (FIG. 4D.2). Based on the phenotype and genotype expression, the CD34⁺ cells differentiated in VEGF₁₆₅ or PDGF_{BB}-supplemented medium were designated by endothelial-like (EL) and smooth muscle-like (SML) cells, respectively.

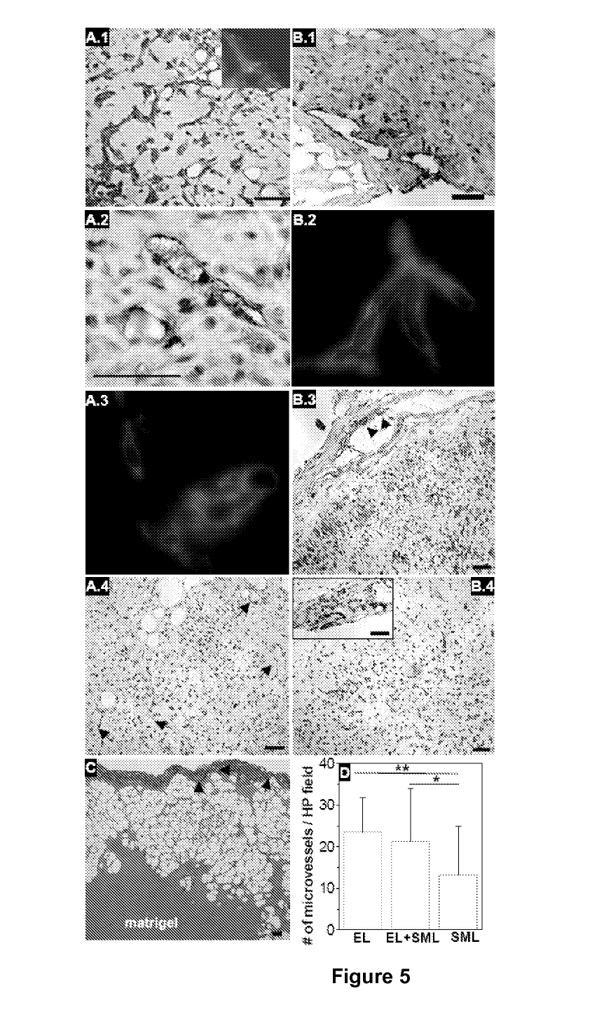

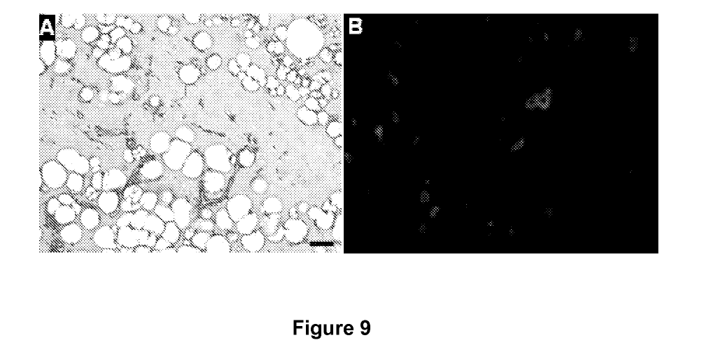

Transplantation of EL and SML cells into nude mice resulted in formation of microvessels. EL or SML cells alone or EL mixed with SML cells (3:1 ratio) were suspended in Matrigel and injected subcutaneously in the dorsal region of nude mice. The implants were removed and analysed after 28 days. Matrigel implanted in the absence of cells showed no microvessels inside of the matrix, only at the periphery (FIG. 5C). In contrast, the constructs with EL cells showed the presence of microvessels within the Matrigel, most of the vessels (˜95%) having an empty lumen while a small percentage (˜5%, FIG. 5A.2) contained mouse red blood cells. These microvessels were immunoreactive for Ulex europaeus agglutinin-1 (UEA-1, specific for human ECs), anti-human PECAM1 and anti-human collagen type IV (collagen IV is a component of the extracellular matrix actively produced by endothelial cells) (FIG. 5A), indicating that they were composed of human ECs. In general, the cells and microvessels inside Matrigel were not reactive for α-SMA (FIG. 5A.4). Implants formed by a mixture of EL and SML showed the presence of microvessels that were immunoreactive to the same human markers described above (FIG. 5B). A fraction of these microvessels (˜5-6%) contained mouse blood cells (FIG. 5B.3). In addition, certain cells inside Matrigel stained positively for α-SMA, and formed small tubules or surrounded human microvessels (FIG. 5B.4). Thus, these cells have properties of SM cells. Constructs with only SML cells stained for α-SMA, showing the differentiation of these cells into the smooth muscle-cell lineage (FIG. 9). These constructs also present microvessel lumens that were immunoreactive for anti-human collagen IV (FIG. 9) indicating that these cells may also differentiate into ECs in vivo. The number of microvessels assessed by antihuman collagen type IV was significantly lower than those found within constructs containing EL cells (FIG. 5D).

### Discussion

In various embodiments, the present inventions provide a procedure to isolate vascular progenitor cells from hESCs that give rise to both EL and SML cells under specific differentiation conditions. In various embodiments, methods are provided for isolating higher yields of vascular progenitor cells than previously reported (10% versus 2%) within a short timeframe (10 days versus 13-15 days). This procedure includes three steps: 1. the differentiation of hESCs through EBs for 10 days; 2. the isolation of CD34⁺ cells by immunomagnetic beads; and 3. the culture of these cells in gelatin-coated dishes in the presence of EGM-2 medium enriched with VEGF₁₆₅ or PDGF_{BB}, for EC or SMC differentiation, respectively.

The CD34 marker was selected to isolate vascular progenitor cells for several reasons. First, we believed that CD34⁺ cells from human blood cells could give rise to ECs and SMCs. Second, human EBs express this marker at higher levels than other endothelial markers including KDR/flk-1 and PECAM1. Third, CD34 is up-regulated during differentiation of human EBs, in contrast to KDR/Flk-1, and all the cells that stained positively for PECAM1 upon day 10 co-express CD34. Fourth, CD34⁺ cells form vessel-like structures within EBs.

The composition of differentiation medium exerts an effect on the yield and differentiation of CD34⁺ cells. EBs grown in differentiation medium containing KO-SR yield fewer CD34⁺ cells than EBs grown in differentiation medium containing FBS. Furthermore, CD34⁺ cells isolated from EBs grown in media with FBS co-express higher levels of other endothelial markers than the cells isolated from EBs grown in KO-SR media. The data collected in the course of this example indicates that EBs grown in FBS media differentiate more rapidly than EBs grown in KO-SR media.

The differentiation of CD34⁺ cells, cultured in the presence of VEGF₁₆₅, into EL cells, was confirmed by their morphology, biochemical markers and functional capacity. The levels of VEGF have an effect on the differentiation of CD34⁺ cells into ECs. When CD34⁺ cells are cultured in EGM-2 medium (low levels of VEGF), only ˜26% were seen to express PECAM1 marker after the 1^{st} passage and they start to lose this marker after several passages (FIG. 3E and FIG. 3H). This may indicate that other cell types take over the cell culture likely due to a high proliferation rate, or the starting cells may differentiate into other cell types. It should be noted that only CD34⁺ cells but not CD34⁻ cells express significant levels of endothelial markers when exposed to VEGF-enriched medium, which indicates that medium alone is not sufficient for the differentiation of hESCs into the vascular cell lineage.

SMC markers such as α-SMA and SM-MHC are still expressed in CD34⁺ cells cultured in EGM-2 medium containing VEGF (FIG. 3). These markers were present on CD34⁺ cells when first isolated from EBs and still expressed after cell differentiation in VEGF-enriched medium, however to a lower extent (specifically for SM-MHC marker) (FIGS. 2 and 3). This indicates that the differentiation process of EL cells was not completed and down-regulation of typical SMC markers occurs over time. Co-expression of endothelial and SMC markers has been previously reported in ECs at several stages during in vitro culture or in vivo differentiation. Our data suggests that hES-derived endothelial cells lose SMC markers after transplantation in nude mice for 28 days.

The CD34⁺ cells cultured in EGM-2 medium containing PDGF_{BB} for 3 passages showed minimal expression of EC markers but significant expression of SMC markers. The expression of SMC markers (particularly SM-MHC) increased comparatively to cells grown in EGM-2 supplemented with 50 ng/mL of VEGF₁₆₅ but not in cells grown in EGM-2 alone. Up-regulation of SMα-22, caldesmon, and angiopoietin 1, known markers for maturing SMCs, was achieved only in differentiating CD34⁺ cells in PDGF-enriched medium. In addition, these cells seem functionally different from those differentiated in EGM-2 medium or VEGF-supplemented EGM-2 medium since they rarely form cord-like structures on matrigel. Our data also indicates that the differentiation of SML is not complete since these cells express a low percentage of PECAM1 (˜5%) and CD34 (˜1%) markers and express genetopically Tie2 and angiopoietin2 markers known to be displayed by ECs.

The transplantation of EL alone or EL with SML into nude mice, using Matrigel as scaffold, contributed to the formation of human microvessels (FIG. 5). In some cases, these microvessels contained mouse blood cells, indicating that these vessels may anastomosize with the host vasculature (FIGS. 5A.2 and 5B.3). The number of microvessels observed in constructs with both EL and SML cells was not statistically different from the ones observed in the absence of SML. However, when SML cells were used in the constructs, α-SMA⁺ cells were observed, thus showing that the SML cells can mature in vivo into smooth muscle cells.

### Example 2

### Cell Genetic Integrity and Production of Functional SML Cells

In this example, further data is presented on the various embodiments of the present invention that obtain from human embryonic stem cells (hESCs) a population of vascular progenitor cells that have the ability to differentiate into endothelial-like (EL) and smooth muscle-like (SML) cells. This example presents data showing that in various embodiments that the EL and SML cells so obtained retain their genetic integrity and/or functionality.

This example shows, in part, that using various embodiments of the present inventions that cells isolated from EBs at day 10 and expressing the hematopoietic/endothelial marker CD34 are vascular progenitor cells that can be selectively induced to differentiate into either endothelial-like (EL) (using endothelial growth medium (EGM-2) containing VEGF₁₆₅), or smooth muscle-like (SML) cells (using EGM-2 medium containing PDGF_{BB}).

In addition, this example presents implantation studies using various embodiments of the methods of the present inventions. When implanted in nude mice, these cells contributed to the formation of functional microvessels containing mouse blood cells. The implantation studies in nude mice show that both cell types (EL and SML) contribute to the formation of human microvasculature. Some microvessels contained mouse blood cells, which indicates functional integration with the host vasculature. Therefore, the vascular progenitors isolated from hESC using various embodiments of the methods of the present inventions provide in various embodiments a vascular tissue engineering construct comprising EL and/or SML cells produced according to the present inventions disposed in and/or on a support substrate.

### Materials and Methods

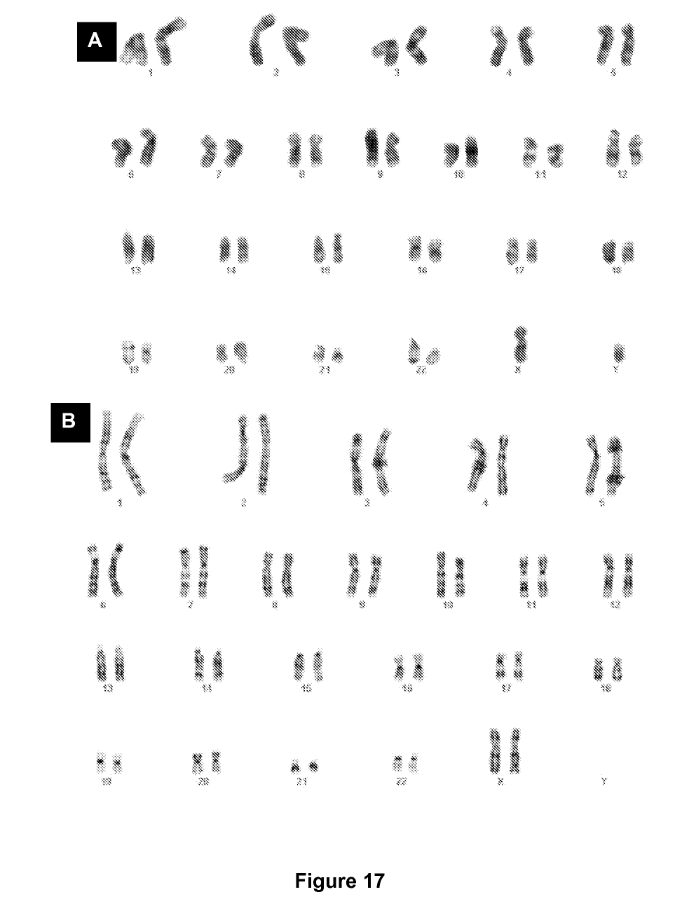

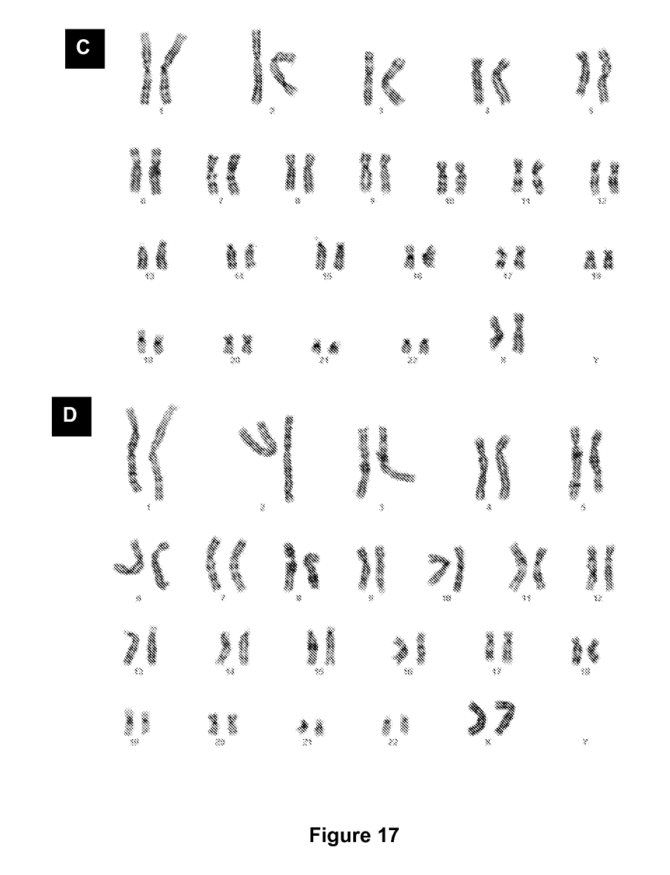

Cell culture. Human ESC lines H9 and H13 with normal karyotype (FIGS. 17A-B) were grown (passages 25 to 45; WiCell, Wisconsin) on an inactivated mouse embryonic feeder layer (MEF, Cell Essential, Boston, Mass.). The studies were performed with H9 cell line unless otherwise stated. In some cases, CD9⁺GCTM2⁺ cells isolated by fluorescence-activated cell sorting (FACS) from hESCs were used to characterize the undifferentiated fraction of these cells. To induce the formation of human EBs, undifferentiated hESCs were treated with 2 mg/mL type IV collagenase (Invitrogen) for 2 h and then transferred (2:1) to low attachment plates (Ø=10 cm, Ref: 3262, Corning) containing 10 mL of differentiation medium [80% Knockout-Dulbecco's Modified Eagle Medium (Invitrogen), 20% Knockout-serum (KO-SR, Invitrogen) or fetal bovine serum (FBS, Hyclone), 1 mM L-glutamine, 0.1 mM β-mercaptoethanol and 1% nonessential amino acid stock (all from Invitrogen)]. EBs were cultured for 12 days at 37° C., and 5% CO₂ in a humidified atmosphere, with media changes performed every 3-4 days. To serve as controls, human vascular smooth muscle cells (hVSMCs) and human umbilical vein endothelial cells (HUVECs) were obtained from Cambrex and cultured in EGM-2 or SmGM-2 media (Cambrex). Medium was changed every other day.

Isolation and culture of CD34⁺ cells. Selection of CD34⁺ cells at day 10 was performed by labeling the hESCs with the anti-CD34 antibody (QBEND/10, Miltenyi Biotec) conjugated with magnetic beads. The magnetically labeled cells were separated into CD34⁺ and CD34⁻ populations using a LS-MACS column (Miltenyi Biotec). CD34 enrichment was confirmed by flow cytometry analysis using a different anti-CD34 antibody (AC136, Miltenyi Biotec). Isolated CD34⁺ cells were grown on 24-well plates (3×10⁴ cells/well) coated with 1% gelatin and containing EGM-2 medium, or EGM-2 medium supplemented with VEGF₁₆₅ (50 ng/mL, R&D Systems) or PDGF_{BB} (50 ng/mL, R&D Systems).

Transmission electron microscopy. Cells seeded in Matrigel-coated 24-well plate were fixed for 1 h in 2.5% (w/v) glutaraldehyde, 3% (w/v) paraformaldehyde, and 5.0% (w/v) sucrose in 0.1 M sodium cacodylate buffer (pH 7.4) and then post-fixed in 1% (w/v) OsO₄ in veronal-acetate buffer for 1 h. The cells were stained en bloc overnight with 0.5% uranyl acetate in veronal-acetate buffer (pH 6.0), dehydrated, and embedded in Spurrs resin. Sections were cut on a Reichert Ultracut E at a thickness of 70 nm with a diamond knife. Sections were examined with a Philips EM410 electron microscope.

Immunostaining. For staining, EBs were transferred to gelatin-coated cover slips with differentiation medium containing 20% (v/v) fetal bovine serum (FBS), allowed to attach overnight, and then, fixed with 4% (w/v) paraformaldehyde for 30 minutes at room temperature. For the evaluation of SMC or EC phenotypes in differentiated CD34⁺ cells a similar fixation procedure was adopted. After blocking with 3% BSA solution, the cells were stained for 1 h with the following anti-human primary antibodies: PECAM1 (JC70A), CD34 (QBEnd 10), vWF (F8/86), α-SMA (1A4), SM-MHC (SMMS-1), calponin (CALP) (all from Dako) or VE-cad (F-8; Santa Cruz Biochemicals). In each immunofluorescence experiment, an isotype-matched IgG control was used. Binding of primary antibodies to specific cells was detected with anti-mouse IgG Cy3 conjugate (Sigma). Cell nuclei were stained with 4′,6-diamidino-2-phenylindole (DAPI) or Topro-3 (Sigma) Immunostaining was examined with either a fluorescence microscope (Nikon) or Zeiss LSM 510 confocal microscope.

For uptake of Dill-labelled acetylated low-density lipoprotein (ac-LDL), differentiated CD34⁺ cells were incubated with 10 mg/mL Dill-labelled ac-LDL (Biomedical Technologies) for 4 h at 37° C. After incubation, cells were washed three times with PBS, fixed with 4% (w/v) paraformaldehyde for 30 min and visualized with a fluorescent microscope.

Histological examination. Immunohistochemical staining of explants from animal study was carried out using the Dako EnVision™+/HRP kit (Dako) with prior heat treatment at 95° C. for 20 min in ReVeal buffer (Biocare Medical) or trypsin (1 mg/mL) for epitope recovery. For immunofluorescent staining, anti-mouse IgG Cy3 conjugate was used as secondary antibody followed by DAPI nuclear staining. The primary antibodies were anti-human PECAM1 (1:20), anti-human collagen type IV (1:500, Sigma), anti-α-smooth muscle actin (α-SMA) (1:50), anti-human nuclei (1:20, Chemicon), β₂-microglobulin (1:50, BD Pharmingen) and the corresponding isotype controls. Biotinylated Ulex europaeus agglutinin-1 (UEA-1, 1:100, Vector Laboratories) was also used for histological staining. The number of microvessels that were immunoreactive for human collagen type IV was counted in 7 random fields from at least four implants (2 sections for each implant) at ×20 magnifications (corresponding to an area of 3.4×10⁵ μm²).

Matrigel assay. For Matrigel differentiation assay, a 24-well plate was coated with 0.4 mL of Matrigel per well and incubated for 30 minutes at 37° C. CD34⁺ cells differentiated in EGM-2 medium, or EGM-2 medium supplemented with VEGF₁₆₅ or PDGF_{BB}, for 3 passages, were seeded on top of the Matrigel at a concentration of 2.5×10⁴−1×10⁵ per 300 μL of culture medium. After 1 h of incubation at 37° C., 1 mL of medium was added. Cord formation was evaluated by contrast-phase microscopy 24 or 48 h after seeding the cells.

Fluorescence-activated cell sorting (FACS) analysis. Undifferentiated hES, HUVEC or CD34⁺ cells grown in different growth media were dissociated with non-enzymatic cell dissociation solution (Sigma) for 10 min. EBs were dissociated with 0.4 U/mL collagenase B (Roche Diagnostics) for 2 h in a 37° C. incubator, followed by treatment with cell dissociation solution for 10 min, followed by gentle pipetting. Single cells were aliquoted (1.25−2.5×10⁵ cells were used per condition) and stained with either isotype controls or antigen-specific antibodies. Single cells were aliquoted (1.25−2.5×10⁵ cells were used per condition) and stained with either isotype controls or antigen-specific antibodies: SSEA-4-PE (MC813-70, R&D Systems), PECAM1-FITC (30884X, BD Pharmingen), CD34-PE/CD34-FITC (AC136, Miltenyi Biotec), KDR/Flk1-PE (89106, R&D) and CD45-FITC (HI30, BD Pharmingen). Cells were analyzed without fixation on a FACScan (Becton Dickinson) using propidium iodide to exclude dead cells. For α-SMA, SM-MHC (all from Dako) and alkaline phosphatase-APC (R&D systems) markers, an intrastain kit (Dako) was used for the fixation and permeabilization of cell suspensions. In case of α-SMA and SM-MHC, the monoclonal antibodies were conjugated with a FITC-secondary antibody (Dako). Data analysis was carried out using CellQuest software.

Western Blot Analysis. Cells differentiated for three passages were harvested using trypsin and lysed. Briefly, sample loading buffer and reducing agent (both from Biorad) were added to the lysates. Samples were heated (5 mM, 95° C.) and loaded on 4-15% Tris-HCl-Criterion gels (Biorad), separated by SDS-PAGE and transferred to nitrocellulose. Membranes were probed for smooth muscle myosin heavy chain (SM-MHC) (8.5 ng/ml, DakoCytomation), α-SMA (0.7 ng/ml, DakoCytomation) and PECAM-1 (2 ng/ml, Santa Cruz Biotechnology).Blots were blocked (30 min), incubated in primary antibody in block (1 h, Pierce), rinsed three times in 10 mM Tris-base/150 mM NaCl/0.1% Tween20 (TBST), pH 7.6, incubated in appropriate horseradish peroxidase-conjugated secondary antibody (anti-mouse IgG or anti-rabbit IgG, 1:1500, Cell Signaling) in block (1 h), and rinsed three times (TBST). Blots were developed using enhanced chemiluminescent kits (Amersham) and exposed to BioMax XAR film (Kodak). Blots were similarly reprobed for glyceraldehyde-3-phosphate dehydrogenase (GAPDH) (2 ng/ml, Santa Cruz Biotechnology).

Reverse Transcription-Polymerase Chain Reaction (RT-PCR) analysis. Total RNA was extracted using trizol (Invitrogen) according to manufacturer's instructions. Total RNA was quantified by a UV spectrophotometer, and 1 ng was used for each RT sample. RNA was reversed transcripted with M-MLV and oligo (dT) primers (Promega) according to manufacturer's instructions. PCRs were done with BIOTAQ DNA Polymerase (Bioline) using 1 μL of RT product per reaction. To ensure semi-quantitative results of the RT-PCR assays, the number of PCR cycles for each set of primers was verified to be in the linear range of the amplification. In addition, all RNA samples were adjusted to yield equal amplification of glyceraldehyde-3-phosphate dehydrogenase (GAPDH) as an internal standard. Primer sequences, reaction conditions, and optimal cycle numbers are given in Table 2. The amplified products were separated on 2% agarose gels with ethidium bromide.

Statistical analysis. An unpaired Student t test or one-way analysis of variance with Bonferroni post test were performed for statistical tests by using GraphPad Prism 4.0 (San Diego, Calif.). Results in this example were considered statistically significant when P 0.05.

Table 2 presents information on primer sequences, various reaction conditions and optimal cycle numbers used for the RT-PCR analyses of vascular markers for various gene transcripts in this example. For the data of Table 2, unless the gene transcript is marked with the notation ***, the PCR conditions comprised the following: 5 minutes at 94° C. (hot start), 30 to 40 cycles (actual number noted in the table); 94° C. for 30 seconds, annealing temperature (noted in the table) for 30 seconds; 72° C. for 30 seconds. A final 7 minutes extension at 72° C. was performed at the end. For the gene transcripts with the notation ****, the PCR conditions comprised the following: 15 minutes at 95° C., 1 minute at 94° C., annealing temperature for 1 minute, 72° C. for 1 minute. A final 10 minutes extension at 72° C. was performed at the end

TABLE 2

| Gene | | Product | | Annealing | [MgCl₂] |
|---|---|---|---|---|---|
| transcript | Primer sequences (5′ to 3′, F_{w}/Rᵥ) | (bp) | Cycles | temp. (° C.) | (mM) |
| PECAM1**** | GCTGTTGGTGGAAGGAGTGC | 620 | 28 | 55 | 1.5 |
| | GAAGTTGGCTGGAGGTGCTC | | | | |
| CD34**** | TGAAGCCTAGCCTGTCACCT | 200 | 30 | 55 | 1.5 |
| | CGCACAGCTGGAGGTCTTAT | | | | |
| KDR/Flk-1 | CTGGCATGGTCTTCTGTGAAGCA | 790 | 35 | 60 | 1.5 |
| | AATACCAGTGGATGTGATGGCGG | | | | |
| Angiopoietin-1 | GGGGGAGGTTGGACTGTAAT | 362 | 35 | 60 | 1.5 |
| | AGGGCACATTTGCACATACA | | | | |
| Angiopoietin-2 | GGATCGGGGAGAGAGGAAC | 535 | 35 | 60 | 1.5 |
| | CTCTGCACCGAGTCATCGTA | | | | |
| Tie2 | ATCCCATTTGCAAAGCTTCTGGCTGGC | 512 | 35 | 60 | 1.5 |
| | TGTGAAGCGTCTCACAGGTCCAGGATG | | | | |
| VE-cad | ACGGGATGACCAAGTACAGC | 596 | 35 | 60 | 1.5 |
| | ACACACTTTGGGCTGGTAGG | | | | |
| Von Willebrand | ATGTTGTGGGAGATGTTTGC | 656 | 40 | 55 | 1.0 |
| Factor (vWF) | GCAGATAAGAGCTCAGCCTT | | | | |
| SM-MHC | GGACGACCTGGTTGTTGATT | 670 | 35 | 60 | 1.5 |
| | GTAGCTGCTTGATGGCTTCC | | | | |
| α-SMA | CCAGCTATGTGAAGAAGAAGAGG | 965 | 35 | 60 | 1.5 |
| | GTGATCTCCTTCTGCATTCGGT | | | | |
| Caldesmon | AACAACCTGAAAGCCAGGAGG | 530 | 35 | 60 | 1.5 |
| | GCTGCTTGTTACGTTTCTGC | | | | |
| SMα-22 | CGCGAAGTGCAGTCCAAAATCG | 928 | 35 | 60 | 1.5 |
| | GGGCTGGTTCTTCTTCAATGGGG | | | | |
| GAPDH | AGCCACATCGCTCAGACACC | 302 | 27 | 60 | 1.5 |
| | GTACTCAGCGCCAGCATCG | | | | |

### Results

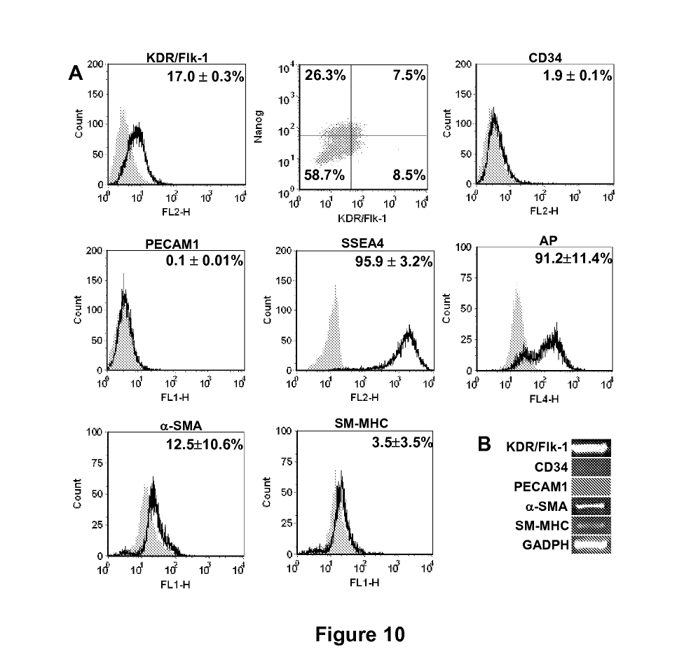

Vascular differentiation during EB development: effects of serum supplements. EBs were grown in medium containing knockout-serum (KO-SR) or fetal bovine serum (FBS) and analysed over a two week period for expression of well-characterized EC (PECAM1, CD34 and KDR/Flk-1) SMC (α-SMA and SM-MHC) and undifferentiated embryonic stem cell markers [SSEA4, Nanog and alkaline phosphatase (AP)] at the gene and protein levels. Initially, hESCs expressed low or undetectable levels of CD34 and PECAM1, significant levels of KDR/Flk-1, and moderate levels of α-SMA and SM-MHC (FIGS. 10A and 10B). The expression of KDR/Flk-1 coexisted with the expression of undifferentiated stem cell markers Nanog (FIG. 10A), SSEA4 and AP, showing that cells are undifferentiated.

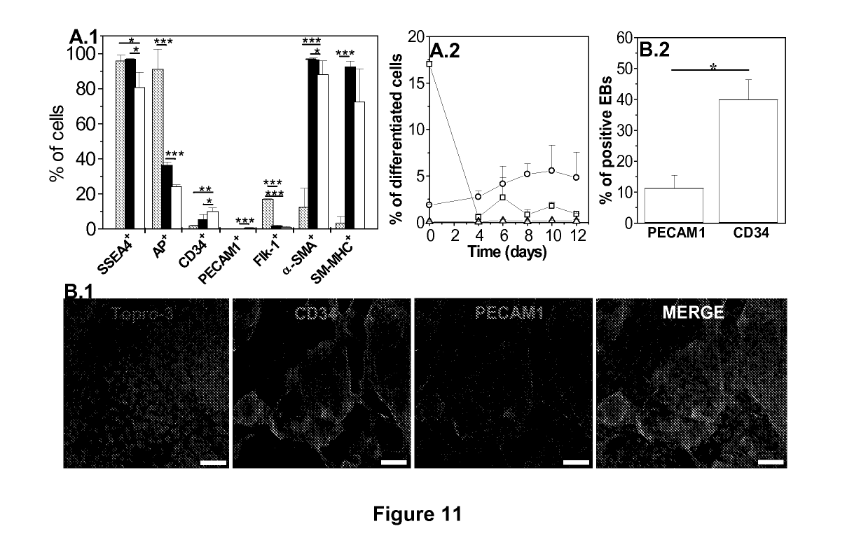

The removal of undifferentiated hESCs from MEFs and subsequent culture as EBs in differentiation medium containing KO-SR reduced the expression of AP and SSEA4, indicating that cells were undergoing differentiation (FIG. 11A.1). During this differentiation process, α-SMA and SM-MHC were highly expressed for 10 days (FIG. 11A.1), expression of CD34 peaked around day 10, KDR/Flk-1 expression decreased by day 4 and remained low thereafter, and PECAM1 expression was low through the 12 days of differentiation (FIG. 11A.2).

The effect of serum supplementation on EB differentiation was also evaluated. The use of FBS instead of KO-SR resulted in a slightly accelerated differentiation process, as indicated by the further decrease of AP and SSEA4 levels and a significant (P<0.05) increase in the expression of CD34 (FIG. 11A.1). EBs grown in medium containing FBS showed lower expression of α-SMA and SM-MHC than EBs grown in medium containing KO-SR. Taken together, medium supplementation with FBS enhanced the vascular differentiation of cells in EBs and contributed to high yields of CD34⁺ cells.

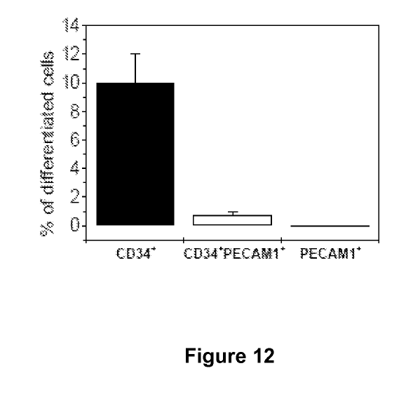

Formation of vessel-like structures in EBs. Confocal analysis of EBs cultured for 10 days showed that CD34⁺ cells formed extensive vascular networks (FIG. 11B.1). The vessel-like structures resemble ones we previously observed in PECAM1⁺ cells (Levenberg S, Golub J S, Amit M, Itskovitz-Eldor J, Langer R. Endothelial cells derived from human embryonic stem cells. Proc Natl Acad Sci USA. 2002; 99:4391-6.); however, these structures were more frequent for CD34⁺ than for PECAM1⁺ cells (FIGS. 11B.1 and 11B.2). FACS analysis confirmed that all PECAM1⁺ cells co-expressed CD34 (FIG. 12).

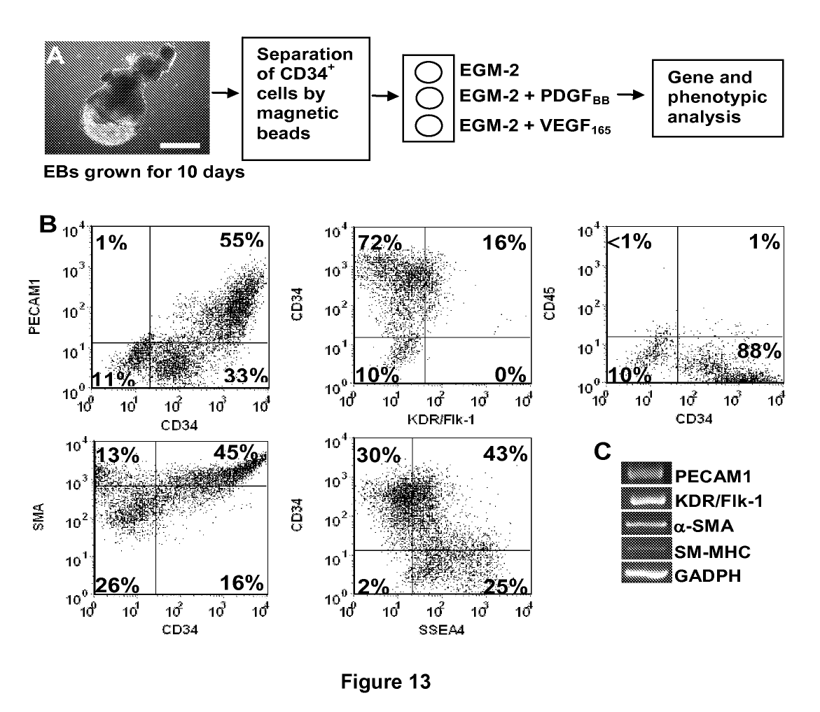

Isolation of CD34⁺ cells. CD34 marker was used to isolate vascular progenitor cells by magnetic selection from EBs grown in differentiation medium with FBS for 10 days (FIG. 13A). These conditions were selected because of high expression of CD34 during EB development (FIGS. 11A.1 and 11A.2). The cells isolated were 92.5±6.7% (n=3) pure for CD34 antigen (approximately a nine fold enrichment of the initial cell population). At this stage, CD34⁺ cells co-expressed high levels of PECAM1 (˜55%), α-SMA (˜45%) and SSEA4 (˜43%), moderate levels of KDR/Flk-1 (˜16%) and low levels of the hematopoietic marker CD45 (˜1%) (FIG. 13B). The presence of these markers was also confirmed at the gene level (FIG. 13C).

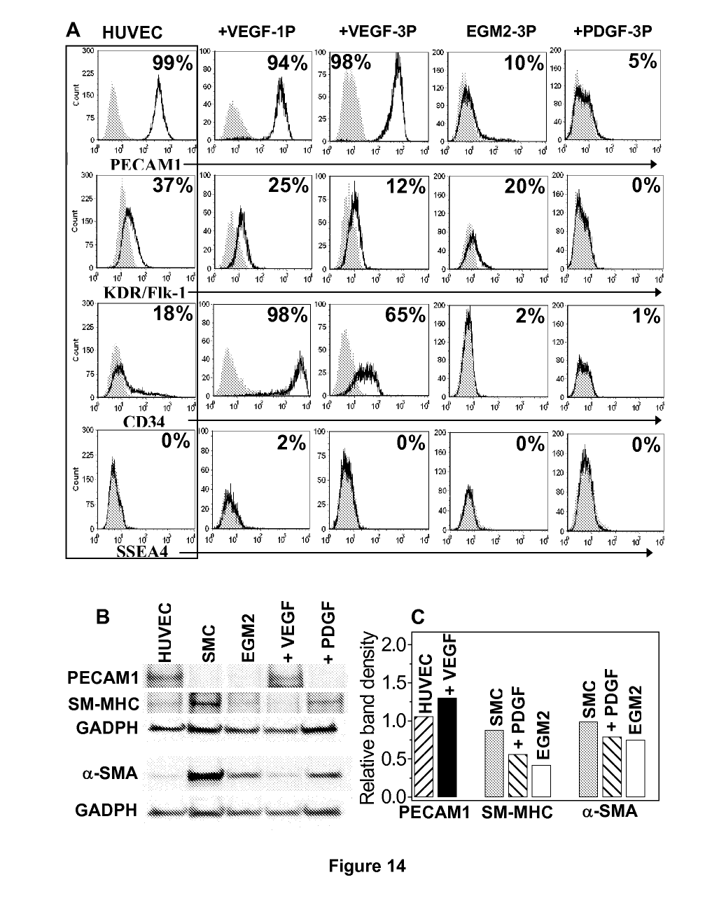

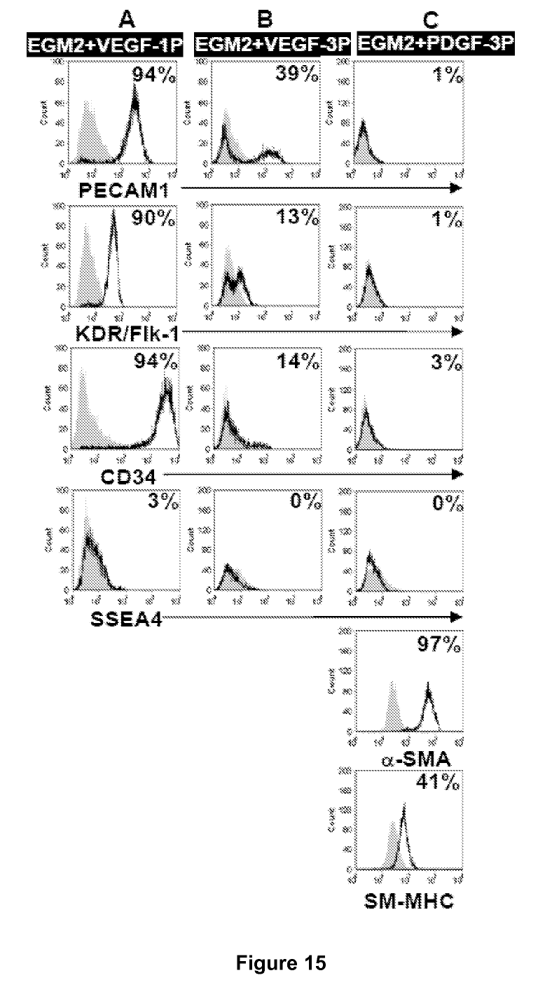

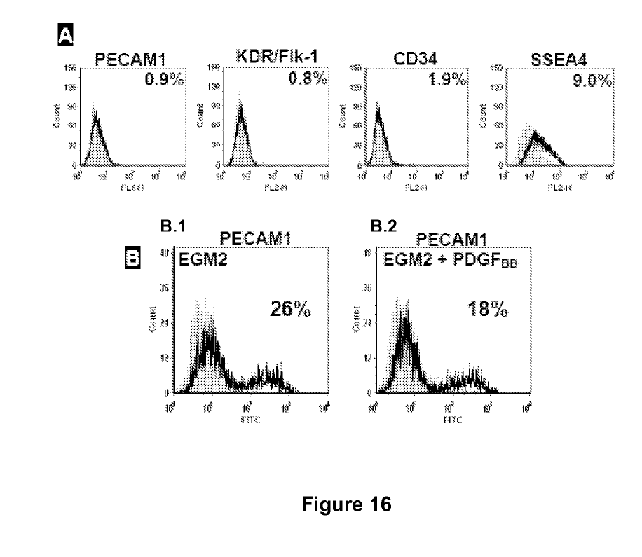

Induction of CD34⁺ cell differentiation into endothelial and smooth muscle cell lineages. The isolated CD34⁺ cells were cultured with EGM-2 medium alone or medium supplemented with VEGF₁₆₅ (50 ng/mL) or PDGF_{BB} (50 ng/mL) (FIG. 13A). CD34⁺ cells cultured in VEGF-supplemented EGM-2 medium for 1 passage (10-15 days after cell seeding) expressed high levels of endothelial cell markers (FIG. 14A). Similar results were obtained with H13 cell line (FIG. 15). As compared to human umbilical vein endothelial cells (HUVECs), CD34⁺ cells had slightly lower expression of PECAM1 and KDR/Flk-1 (FIG. 14A), and higher expression of CD34. At this stage, the cells lost nearly all expression of the marker SSEA4, indicating their differentiated state. CD34⁻ cells grown in the same conditions as CD34⁺ cells showed minimal expression of the endothelial markers (FIG. 16), indicating that CD34⁺ cells, but not the CD34⁻ cells can be effectively induced toward an endothelial lineage. CD34⁺ cells cultured in EGM-2 medium or EGM-2 medium supplemented with PDGF_{BB} for 1 passage showed a much lower expression of PECAM1 (26% and 18%, respectively) than the CD34⁺ cells cultured in VEGF-supplemented medium (94%) (FIG. 16). As EGM-2 medium contains <5 ng/ml VEGF₁₆₅ (as measured by an ELISA kit), VEGF concentration has an effect on the endothelial differentiation of CD34⁺ cells.

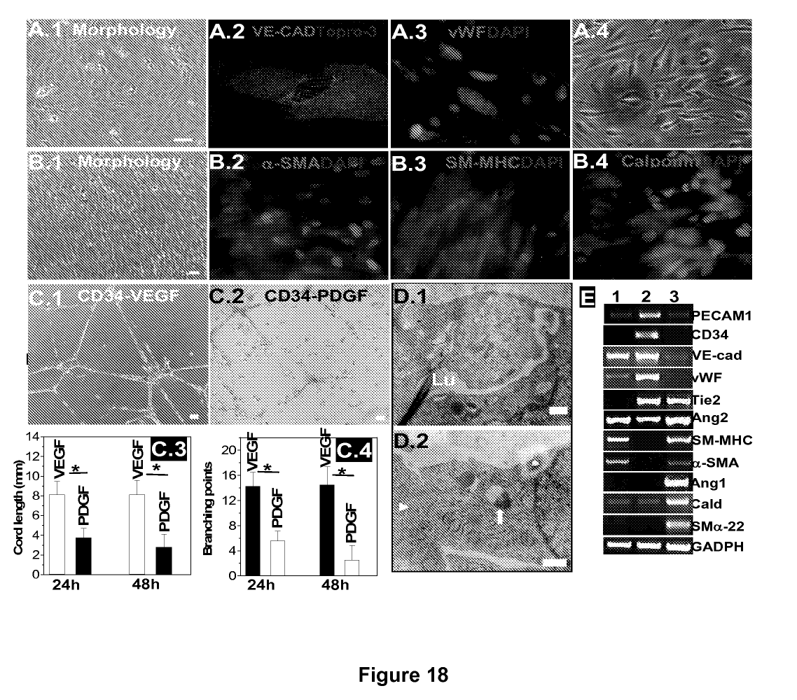

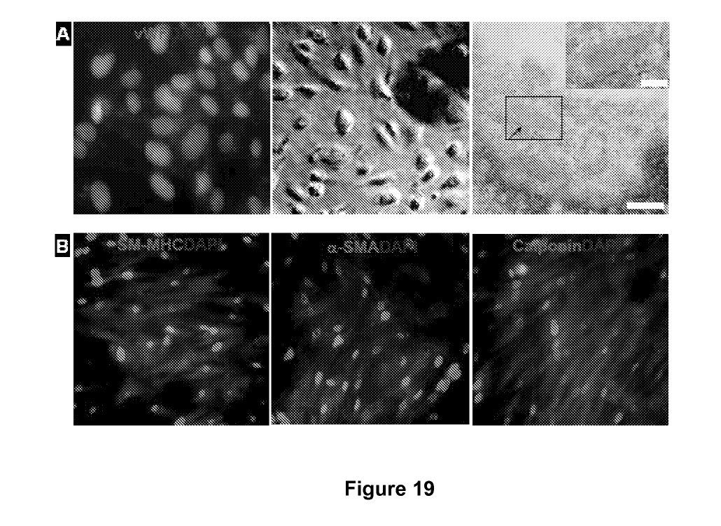

The proliferation rate of CD34⁺ cells cultured in VEGF-supplemented medium is high, achieving 20 population doublings over a two-month period. FACS analyses of CD34⁺ cells cultured for 3 passages (FIG. 14A) showed the expression of PECAM1 comparable to that in HUVEC (similar results were obtained by Western Blot; FIGS. 14B and 14C), albeit different regarding the expression of CD34 and KDR/Flk-1 markers. Karyotyping analyses showed that genetic integrity was preserved during differentiation (FIG. 17). Differentiated CD34⁺ cells stained positively for VE-cadherin at cell-cell adherent junctions, produced vWF, and were able to incorporate ac-LDL (FIG. 18A), typical markers found in endothelial cells (FIG. 19). Genetic analysis demonstrated that these cells express PECAM1, CD34, VE-cadherin, vWF and Tie2 receptor, but are negative for SMC markers including SM-MHC, SMα-22 and angiopoietin-1 (FIG. 18E). CD34⁺ cells isolated from H13 cell line and differentiated in VEGF-supplemented medium presented lower levels of PECAM1 (39% versus 98%) and CD34 (14% versus 65%) compared to the H9 cell line (FIG. 15), suggesting slightly different differentiation profiles in the two cell lines.

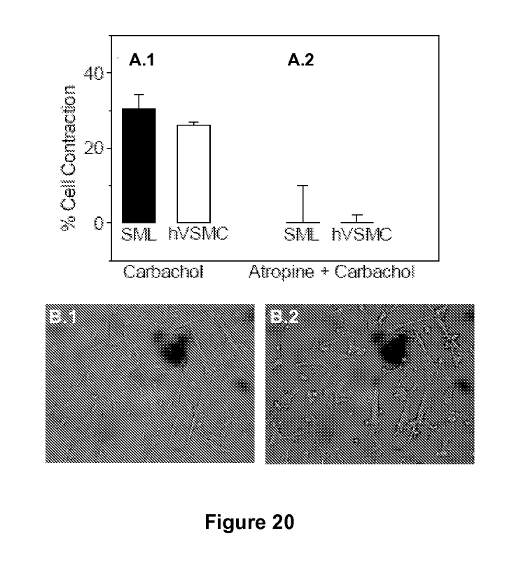

Cells cultured in EGM-2 medium or PDGF_{BB}-supplemented medium for 3 passages expressed high levels of α-SMA, SM-MHC and calponin (FIGS. 14 and 18B), low levels of endothelial markers 20%), and no detectable expression of the undifferentiating stem cell marker SSEA4. Western blot analysis showed that expressions of SM-MHC and α-SMA were higher in cells differentiated in EGM2 supplemented with PDGF_{BB} (FIGS. 14B and 14C). As confirmed by RT-PCR (FIG. 18E), PDGF_{BB}-supplemented EGM-2 medium upregulated the expression of definitive SMC markers including caldesmon and SMα-22, and the expression of angiopoietin-1, a ligand produced by SMCs that activates the receptor Tie-2 found on ECs. This indicates that the presence of PDGF_{BB} contributed to cell maturation towards SMC phenotype. However, this process is not complete since cells express the endothelial markers angiopoietin-2 and Tie2. To examine whether these smooth muscle-like cells were functional, they were subjected to the effects of carbachol and atropine (FIG. 20). After exposure to carbachol 10⁻⁵ M the cells contracted 30% after 30 min. In addition, the muscarinic antagonist atropine was shown to block the carbachol-mediated effects. Similar results were obtained in human vascular smooth muscle cells (hVSMCs). CD34⁺ cells grown in the presence of PDGF had higher proliferation rates than CD34⁺ cells grown in the presence of VEGF, with 42 population doublings over a two month period. Karyotyping analyses showed that genetic integrity was preserved during differentiation (FIG. 17).

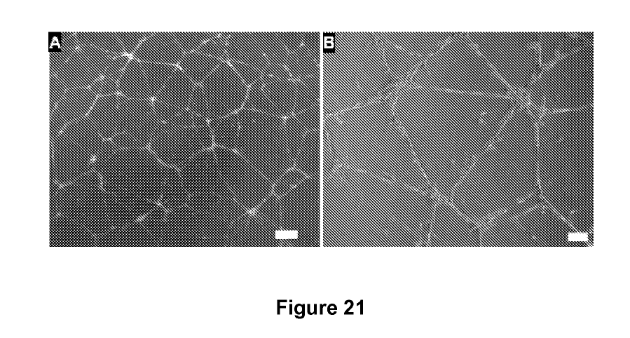

The ability of CD34⁺ cells differentiated in VEGF or PDGF-supplemented medium to form cord-like structures was also assessed by culturing these cells in the extracellular matrix basement membrane, Matrigel. CD34⁺ cells differentiated in VEGF-supplemented medium were able to spontaneously reorganize into cord-like structures when maintained in culture for 24 h (FIG. 18C and FIG. 21). In contrast, CD34⁺ cells differentiated in EGM-2 medium containing PDGF_{BB} have limited ability to form cord-like structures (FIG. 18C). Transmission electron micrographs of cord-sections formed by CD34⁺ cells differentiated in VEGF₁₆₅-supplemented medium showed the presence of a lumen (FIG. 18D.1), thus confirming the capacity of these cells to form vascular networks in vitro. In addition, these cells presented typical endothelial features (FIG. 19) such as the presence of round or rod-shaped structures that resemble Weibel-Palade bodies and tight junctions between cells (FIG. 18D.2). Based on the phenotype and genotype expression, the CD34⁺ cells differentiated in VEGF₁₆₅ or PDGF_{BB}-supplemented medium were designated endothelial-like (EL) and smooth muscle-like (SML) cells, respectively.

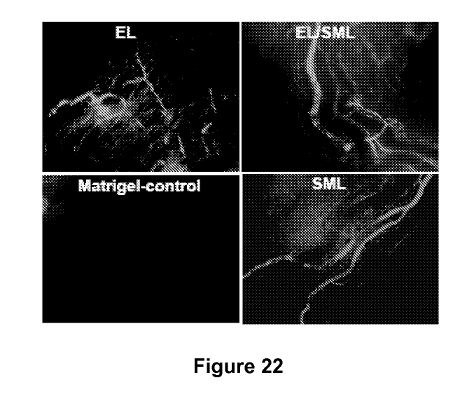

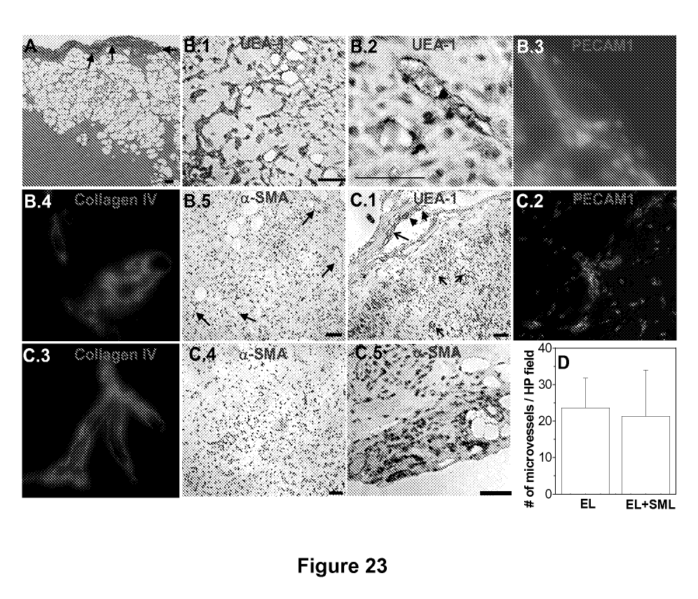

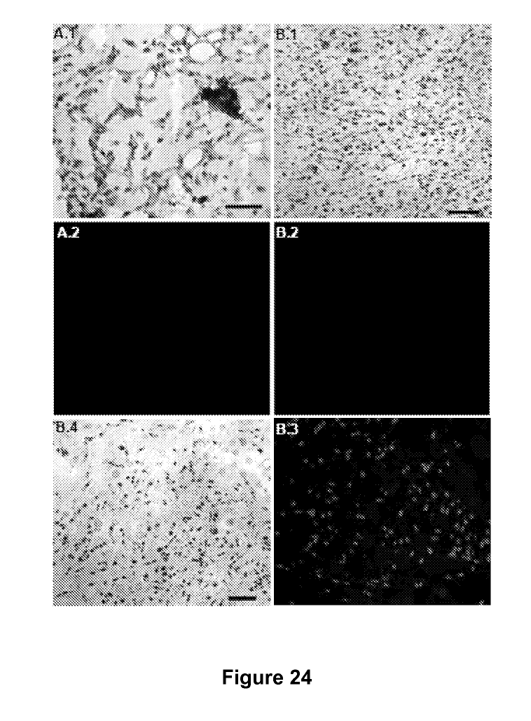

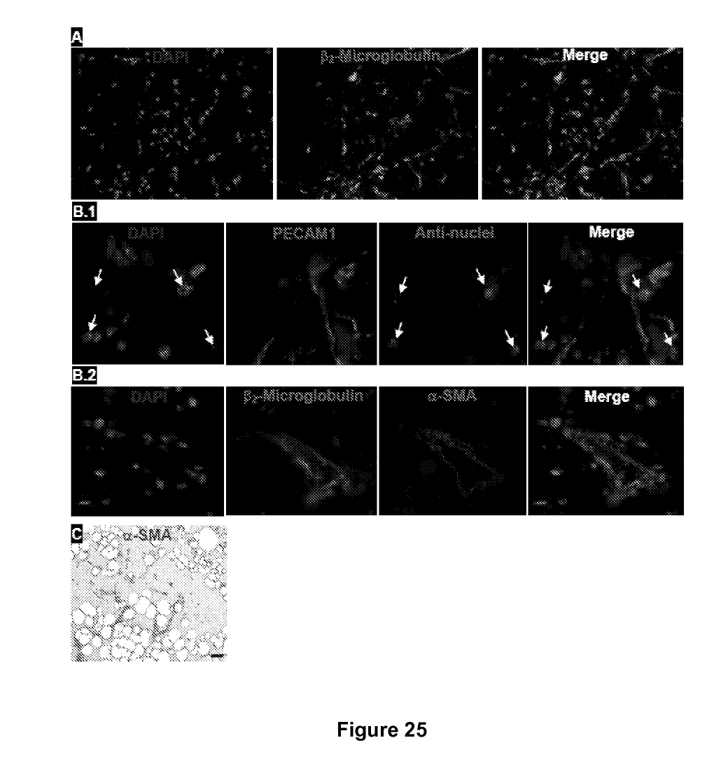

Transplantation of EL and SML cells into nude mice resulted in formation of microvessels. EL or SML cells alone or EL mixed with SML cells (3:1 ratio) were suspended in Matrigel and injected subcutaneously in the dorsal region of nude mice. After 28 days, the mice were injected intravenously with FITC-dextran solution. The Matrigel implants were then removed and imaged. Microvessels that support blood flow were observed in Matrigel implants containing EL or SML cells, but rarely in matrigel without cells (FIG. 22). Matrigel implanted in the absence of cells showed no microvessels inside of the matrix, only at the periphery (FIG. 23A). The constructs with EL cells showed the presence of microvessels within the Matrigel (FIG. 23B.1), most of which (˜95%) were patent with empty lumens while a small percentage (˜5%, FIG. 23B.2) contained mouse red blood cells. These microvessels were reactive for Ulex europaeus agglutinin-1 [UEA-1, specific for human ECs], anti-human PECAM1, anti-human nuclei and anti-human collagen type IV [collagen IV is a component of the extracellular matrix actively produced by endothelial cells] (FIG. 23B and FIGS. 24 and 25), indicating that they were composed of human ECs. In general, the cells and microvessels inside Matrigel were not reactive for α-SMA (FIG. 23B.5). On the other hand, implants formed by a mixture of EL and SML showed the presence of microvessels that were immunoreactive to the same human markers described above (FIG. 23C). A fraction of these microvessels (˜5-6%) contained mouse blood cells (FIG. 23C.1). Cells inside Matrigel stained positively for PECAM1 (˜41%) or α-SMA (˜20%), in this last case they formed small tubules (FIG. 23C.4) or surrounded human microvessels (FIG. 23C.5; FIG. 25). Thus, these cells have properties of SM cells. Constructs with only SML cells stained for α-SMA (FIG. 25) showing the differentiation of these cells into the smooth muscle-cell lineage.

### Discussion

This example illustrates the practice of various embodiments of methods for the isolation and differentiation of vascular progenitor cells from hESCs. The data of this example show that a CD34⁺ population (of 93% purity) contains progenitors that can give rise to both EL and SML cells if cultured according to one or more of the embodiments of the present inventions. In various embodiments, the methods include three steps: (i) the differentiation of hESCs through EBs for 10 days; (ii) the isolation of CD34⁺ cells by immunomagnetic beads; and (iii) the culture of these cells in gelatin-coated dishes in the presence of EGM-2 medium enriched with VEGF₁₆₅ or PDGF_{BB} for EC or SMC differentiation, respectively.

CD34 marker was selected to isolate vascular progenitor cells for several reasons. First, we believed that CD34⁺ cells from human blood cells could give rise to ECs and SMCs. Second, human EBs express this marker at higher levels than other endothelial markers including KDR/flk-1 and PECAM1. Third, CD34 is up-regulated during differentiation of human EBs, in contrast to KDR/Flk-1, and all the cells that stained positively for PECAM1 upon day 10 co-express CD34. Fourth, CD34⁺ cells form vessel-like structures within EBs.

This example shows that the composition of differentiation medium exerts an effect on the differentiation of EBs and yield of CD34⁺ cells. EBs grown in differentiation medium containing KO-SR yield fewer CD34⁺ cells than EBs grown in differentiation medium containing FBS. Furthermore, our data indicates that EBs grown in FBS media differentiate more rapidly than EBs grown in KO-SR media. This agrees with previous studies showing that KO-SR contribute for an increase growth rate of nondifferentiated cells.

The data of this example show that CD34⁺ cells cultured in the presence of VEGF₁₆₅ differentiated into EL cells as confirmed by their morphology, biochemical markers and functional studies. The data of this example shows that the levels of VEGF has an effect on the differentiation of CD34⁺ cells into ECs. To the best of our knowledge, effect has not been previously described by others. When CD34⁺ cells are cultured in EGM-2 medium (low levels of VEGF), only ˜26% express PECAM1 marker after the 1^{st} passage and they start to lose this marker after several passages. This may indicate that other cell types take over the cell culture likely due to a high proliferation rate, or that the starting cells may differentiate into other cell types. It should be noted that only CD34⁺ cells but not CD34⁻ cells express significant levels of endothelial markers when exposed to VEGF-enriched medium, which shows that medium alone is not sufficient for the differentiation of hESCs into the vascular cell lineage. The results of this example also show that the differentiation of CD34⁺ cells into the endothelial lineage is slightly different for H9 and H13 cell lines.

This example also provides data on the transplantation of EL cells into nude mice using Matrigel as support substrate contributed to the formation of human microvessels (FIG. 23). In some cases, these microvessels contained mouse blood cells and supported blood flow, indicating that these vessels anastomosed with the host vasculature.

This example also demonstrates that using various embodiments of the present inventions that CD34⁺ cells can be caused to give rise to SML cells, and that PDGF plays a role in this differentiation process. CD34⁺ cells cultured in EGM-2 medium containing PDGF_{BB} for 3 passages show minimal expression of EC markers but significant expression of SMC markers. The expression of SMC markers was also observed in cells grown in EGM-2 alone. However, the expression of SM-MHC, a later marker in SMC differentiation that is not detected in other cell types, was higher in PDGF conditions. In addition, up-regulation of SMα-22, caldesmon, and angiopoietin-1, known markers for maturing SMCs, was achieved only in differentiating CD34⁺ cells in PDGF-enriched medium. Furthermore, these cells seem functionally different from those differentiated in EGM-2 medium or VEGF-supplemented EGM-2 medium since they rarely form cord-like structures on Matrigel. The data of this example also indicates that the differentiation of SML is not complete since these cells express a low percentage of PECAM1 (˜5%) and CD34 (˜1%) markers and genetopically express Tie2 and angiopoietin-2 markers known to be displayed by ECs. SML cells prepared according to various embodiments of the present inventions in this example demonstrated the ability to contract or relax in response to a variety of pharmacologic agents like SMCs and thus are functional. Furthermore, SML cells prepared according to various embodiments of the present inventions in this example showed preserved genetic integrity after the differentiation process over 3 passages as demonstrated by karyotyping analyses. When SML cells prepared according to various embodiments of the present inventions in this example were transplanted into nude mice, using Matrigel as scaffold, α-SMA⁺ cells were observed, forming either small tubules or surrounding microvessels.

All literature and similar material cited in this application, including, but not limited to, patents, patent applications, articles, books, treatises, and web pages, regardless of the format of such literature and similar materials, are expressly incorporated by reference in their entirety for all purposes. In the event that one or more of the incorporated literature and similar materials differs from or contradicts this application, including but not limited to defined terms, term usage, described techniques, or the like, this application controls.

The section headings used herein are for organizational purposes only and are not to be construed as limiting the subject matter described in any way.

While the present inventions have been described in conjunction with various embodiments and examples, it is not intended that the present inventions be limited to such embodiments or examples. On the contrary, the present inventions encompass various alternatives, modifications, and equivalents, as will be appreciated by those of skill in the art.

The inventions should not be read as limited to the described order or elements unless stated to that effect. It should be understood that various changes in form and detail may be made without departing from the scope of the present inventions. Therefore, all embodiments that come within the scope and spirit of the present teachings and equivalents thereto are claimed.

1. A population of cells derived from embryonic stem cells and expressing CD34, wherein each cell in the population also exhibits at least one characteristic selected from a spindle-shape morphology, expression of at least one of α-SMA, SM-MHC, calponin, caldesmon, angiopoietin 1, and SMα-22, and sparse formation of capillary-like structures when placed in MATRIGEL™.

2. The population of claim 1, wherein at least a portion of the cells exhibit at least two of the characteristics.

3. The population of claim 1, wherein at least a portion of the cells exhibit at least three of the characteristics.

4. (canceled)

5. The population of claim 1, wherein at least a portion of the cells exhibit at least five of the characteristics.

6. (canceled)

7. (canceled)

8. The population of claim 1, wherein at least a portion of the cells exhibit all of the characteristics.

9. A method of promoting development of vascular tissue using embryonic stem cells, comprising:
providing a first population of embryonic stem cells;
contacting the first population with type IV collagenase;
culturing the collagenase contacted first population in differentiation medium;
isolating those cells expressing CD34 to produce a first population of CD34⁺ cells; and
culturing the first population of CD34⁺ cells under predetermined conditions to cause their differentiation into endothelial-like or smooth muscle-like cells.

10. The method of claim 9, wherein at least 5% of the population is isolated.

11. (canceled)

12. The method of claim 9, wherein at least 10% of the population is isolated.

13. The method of claim 9, wherein the predetermined conditions comprise VEGF-supplemented EGM-2 medium.

14. The method of claim 9, wherein the predetermined conditions comprise PDGF-supplemented EGM-2 medium.

15. The method of claim 9, wherein the predetermined conditions comprise IL-3, IL-6, granulocyte-macrophage colony stimulating factor, retinoic acid and granulocyte colony-stimulating factor in a methylcellulose solution.

16. The method of claim 15, wherein the predetermined conditions further comprise one or more of FBS, BSA, and erythropoietin.

17. The method of claim 9, wherein the predetermined conditions comprise one or more of angiopoietin (Ang), ephrin (Eph), fibroblast growth factor (FGF), placental growth factor (PIGF), transforming growth factor β-1 [(TGF)-β1], cytokines, erythropoietin, thrombopoietin, transferring, insulin, stem cell factor (SCF), granulocyte colony-stimulating factor (G-CSF) retinoic acid and granulocyte-macrophage colony stimulating factor (GM-CSF).

18. The method of claim 9, wherein the predetermined conditions comprise implantation into an animal.

19. The method of claim 9, wherein the predetermined conditions comprise combination with a hydrogel and one or more growth factors selected from VEGF, PDGF, angiopoietin (Ang), ephrin (Eph), fibroblast growth factor (FGF), placental growth factor (PIGF), transforming growth factor β-1 [(TGF)-β1], cytokines, erythropoietin, thrombopoietin, transferring, insulin, stem cell factor (SCF), granulocyte colony-stimulating factor (G-CSF) retinoic acid and granulocyte-macrophage colony stimulating factor (GM-CSF) to form a mixture and implantation of the mixture into an animal.

20. The method of claim 9, further comprising implanting the differentiated cells in an animal.

21. The method of claim 9, further comprising combining the differentiated cells with a gel.

22. The method of claim 21, wherein the gel comprises one or more of MATRIGEL™, alginate, agarose, and collagen-GAG.

23. The method of claim 22, wherein the gel further comprises a member of the group consisting of collagen I, collagen IV, laminin, fibrin, fibronectin, proteoglycans, glycoproteins, glycoaminoglycans, proteinases, collagenases, chemotactic agents, growth factors, and any combination of the above.

24. The method of claim 21, further comprising combining the cell-gel mixture with a three-dimensional support matrix such that the gel coats internal and external surfaces of the matrix.

25. The method of claim 9, further comprising seeding the differentiated cells on a three dimensional cell support matrix.

26. The method of claim 9, further comprising:
providing a second population of embryonic stem cells
contacting the second population with type IV collagenase;
culturing the collagenase contacted second population in differentiation medium;
isolating those cells expressing CD34 to produce a second population of CD34⁺ cells; and
culturing the second population of CD34⁺ cells under predetermined conditions to cause their differentiation into smooth muscle-like cells, wherein the first population of CD34⁺ cells is cultured under predetermined conditions to cause their differentiation into endothelial-like cells.

27. The method of claim 26, further comprising implanting the endothelial-like cells and the smooth muscle-like cells in an animal.

28. The method of claim 26, further comprising combining the endothelial-like cells and the smooth muscle-like cells with a gel.

29. The method of claim 28, wherein the gel comprises one or more of MATRIGEL™, alginate, agarose, and collagen-GAG.

30. The method of claim 29, wherein the gel further comprises a member of the group consisting of collagen I, collagen IV, laminin, fibrin, fibronectin, proteoglycans, glycoproteins, glycoaminoglycans, proteinases, collagenases, chemotactic agents, growth factors, and any combination of the above.

31. The method of claim 28, further comprising combining the cell-gel mixture with a three-dimensional support matrix such that the gel coats internal and external surfaces of the matrix.

32. The method of claim 26, further comprising seeding the endothelial-like cells and the smooth muscle-like cells on a three dimensional cell support matrix.

33. A population of smooth muscle-like cells derived from embryonic stem cells.

34. (canceled)

35. The population of smooth muscle-like cells of claim 34, wherein the cells contract more than about 20% in the presence of carbachol.

36. The population of smooth muscle-like cells of claim 34, wherein the cells contract more than about 20% in the presence of carbachol and relax to a contraction of less than about 2% in the presence of atropine.

37. (canceled)

38. The population of smooth muscle-like cells of claim 33, wherein the embryonic stem cells are human embryonic stem cells.

39. A population of endothelial-like cells derived from embryonic stem cells.

40. (canceled)

41. The population of endothelial-like cells of claim 39, wherein the embryonic stem cells are human embryonic stem cells.

42. (canceled)

43. A method of relieving or preventing a vascular disease or condition in a mammalian subject, the method comprising:
obtaining a population of vascular progenitor cells;
administering said vascular progenitor cells into the subject under conditions suitable for stimulating differentiation of said vascular progenitor cells into endothelial and smooth muscle cells, thereby alleviating said vascular disease or condition.

44. A method of vascularizing a mammalian tissue, the method comprising:
obtaining a population of vascular progenitor cells
contacting said vascular progenitor cells with said mammalian tissue under conditions suitable for stimulating differentiation of said vascular progenitor cells into endothelial and smooth muscle cells, thereby enriching the vascularity of the tissue.

45. A method of obtaining a population of differentiated cells from a population of stem cells, comprising the steps of:
contacting a population of stem cells with a differentiation medium to form a population of embryoid bodies;
extracting from the population of embryoid bodies at least a portion of the cells expressing the CD34 marker to provide a population of CD34⁺ cells;
contacting the population of CD34⁺ cells with one or more growth factors such that at least a portion of the population of CD34⁺ cells differentiate into one or more of endothelial-like cells and smooth muscle-like cells.

46. The method of claim 45, wherein the step of contacting a population of stem cells with a differentiation medium comprises passaging the cells less than about 50 times.

47. (canceled)

48. The method of claim 45, wherein the step of contacting a population of stem cells with a differentiation medium comprises doing so for time period in the range between about 8 to about 15 days.

49. (canceled)

50. The method of claim 45, wherein the step of contacting the population of CD34⁺ cells with one or more growth factors comprises doing so for time period in the range between about 8 to about 15 days.

51. The method of claim 45, wherein the step of contacting the population of CD34⁺ cells with one or more growth factors comprises doing so for time period in the range between about 15 to about 30 days.

52. (canceled)

53. The method of claim 45, wherein the stem cells are embryonic stem cells.

54. The method of claim 53, wherein the stem cells are human embryonic stem cells.

55. The method of claim 45, wherein the step of extracting from the population of embryoid bodies at least a portion of the cells expressing the CD34 marker comprises extracting greater than about 5% of the cells expressing the CD34 marker from the population of embryoid bodies.

56. (canceled)

57. The method of claim 45, wherein the step of extracting from the population of embryoid bodies at least a portion of the cells expressing the CD34 marker comprises extracting greater than about 15% of the cells expressing the CD34 marker from the population of embryoid bodies.

58. The method of claim 45, wherein the step of contacting the population of CD34⁺ cells with one or more growth factors comprises contacting the population of CD34⁺ cells with a growth factor at concentration of greater than about 30 ng/ml.

59. (canceled)

60. The method of claim 45, wherein the step of contacting the population of CD34⁺ cells with one or more growth factors comprises contacting the population of CD34⁺ cells with a growth factor at concentration in the range between about 30 ng/ml to about 100 ng/ml.

61. The method of claim 45, wherein the one or more one or more growth factors comprise one or more of VEGF, PDGF, angiopoietin (Ang), ephrin (Eph), fibroblast growth factor (FGF), placental growth factor (PIGF), transforming growth factor β-1 [(TGF)-β1], cytokines, erythropoietin, thrombopoietin, transferring, insulin, stem cell factor (SCF), granulocyte colony-stimulating factor (G-CSF), retinoic acid and granulocyte-macrophage colony stimulating factor (GM-CSF).

62. The method of claim 45, wherein the one or more growth factors comprise VEGF₁₆₅ and the differentiated cells comprise endothelial-like cells.

63. (canceled)

64. The method of claim 62, wherein the population of CD34⁺ cells is contacted with concentration of VEGF₁₆₅ that is greater than about 30 ng/ml.

65. (canceled)

66. The method of claim 62, wherein the population of CD34⁺ cells is contacted with concentration of VEGF₁₆₅ that is in the range about 30 ng/ml to about 100 ng/ml.

67. The method of claim 45, wherein the one or more growth factors comprise PDGF_{BB} and the differentiated cells comprise smooth muscle-like cells.

68-69. (canceled)

70. The method of claim 67, wherein the population of CD34⁺ cells is contacted with concentration of PDGF_{BB} that is in the range about 30 ng/ml to about 100 ng/ml.

71. The method of claim 45, wherein the karyotype of the one or more of endothelial-like cells and smooth muscle-like cells is substantially preserved relative to the karyotype of the stem cells.

72-86. (canceled)
